(19) United States
(12) Patent Application Publication
(10) Pub. No.: US 2005/0129787 A1
(43) Pub. Date: Jun. 16, 2005

(54) Pharmaceutical compositions and methods for managing connective tissue ailments

(76) Inventor: Howard Murad, Marina del Ray, CA (US)

Correspondence Address: MORGAN LEWIS & BOCKIUS LLP, 1111 PENNSYLVANIA AVENUE NW, WASHINGTON, DC, 20004, US

(21) Appl. No.: 11/045,405

(22) Filed: Jan. 31, 2005

Related U.S. Application Data
(63) Continuation of application No. 10/316,090, filed on Dec. 11, 2002.
Application No. 10/316,090 is a continuation of application No. 10/051,189, filed on Jan. 22, 2002, now Pat. No. 6,676,977.
Application No. 10/051,189 is a division of application No. 09/641,376, filed on Aug. 18, 2000, now Pat. No. 6,358,539.
(60) Provisional application No. 60/150,034, filed on Aug. 20, 1999.

(51) Int. Cl. A61K 35/78; A61K 31/7048; A61K 31/7008; A61K 31/555

(52) U.S. Cl. 424/728; 514/62; 514/184; 514/561; 514/23; 514/560; 514/423; 514/564; 514/27; 514/456; 424/752; 514/567; 424/766; 424/735; 424/736; 424/765; 424/777

(57) ABSTRACT

The present invention relates to compositions and methods for managing connective tissue disorders in a patient, a sugar compound that is converted to a glycosaminoglycan, a primary antioxidant component, at least one amino acid component, at least one transition metal component, at least one moisturizing agent, at least one fatty acid. In a preferred embodiment, the composition for topical administration to the patient's skin further included hydrogen peroxide in an amount sufficient to cleanse the skin.

### CROSS REFERENCE TO RELATED APPLICATION

This application is a continuation-in-part of U.S. patent application Ser. No. 10/051,189, filed Jan. 22, 2002, which is a divisional of U.S. patent application Ser. No. 09/641,376, filed Aug. 18, 2000, which claims the benefit of provisional application No. 60/150,034 filed Aug. 20, 1999.

### FIELD OF THE INVENTION

This application relates to compositions and methods for managing connective tissue disorders.

### BACKGROUND OF THE INVENTION

Connective tissues encompass a group of tissues characterized by large amounts of intercellular substance and relatively few cells. Connective tissues include tendons, ligaments, aponeuroses, blood vessels, nerves, vitreous humor, cartilage, skin, and bone. These tissues are formed of fibrous and ground substance (i.e. glycosaminoglycan) composed of collagen and/or elastin and various cell types including fibroblasts, macrophages, mast cells, plasma cells, melanocytes, and fat cells. Dense connective tissue contains very few cells and a large amount of fibers organized into regular bundles. This type of connective tissue is found mainly in tendons, ligaments, and aponeuroses. Loose connective tissue contains more cells with less organized fibers. This type of connective tissue is found in blood vessels and organs.

The functions of connective tissues are various. They are largely responsible for the cohesion of the body as an organism, of organs as functioning units, and of tissues as structural systems. Connective tissues also serve as defensive mechanisms against infection and in repair of bodily injuries. Connective tissues also act as a means for transporting nutrients to most of the body's organs and for removing wastes from these organs. Further, connective tissues act as the storehouse of the body, storing body fat, maintaining homeostatic mechanisms, and maintaining the balance of water and salt in the body. Connective tissues, particularly the ligaments and tendons, make motion of the body possible.

Connective tissue disorders are caused by many factors including stresses and strains from mechanical forces, disease, aging, and genetic predisposition. These disorders include but are not limited to skin related disorders such as wrinkles, skin ulcerations, dermatomyositis, systematic sclerosis, decubitus disorders, scleroderma, thinning skin, age spots, abnormal pigmentation, leathery skin, yellowish discoloration of the skin, dry skin, stretch marks, loss of skin elasticity, and bed sores; joint related disorders such as inflammatory arthritis, degenerative joint disease, nonarticular rheumatism, and miscellaneous arthritis; vascular and circulatory disorders such as varicose veins, poor circulation, arthritis, dilated blood vessels, and polyarthritis nodosa; coronary disorders such as coronary heart disease, cardiomyopathies, rheumatic fever, and congenital heart disease; neurological disorders such as peripheral neuropathy; as well as disorders affecting multiple systems of the body such as systemic lupus erythematosus, systemic vasculitis, polymyositis, Sjögren's syndrome, and myositis. Indeed, connective tissue afflictions are quite common, presently affecting millions of Americans. Such afflictions can be painful and, in their extreme, debilitating.

Neurological tissues are particularly susceptible to various disorders due to the effects of biological and environmental factors as well as aging. These neurological disorders include senility, dementia, Alzheimer's disease, peripheral neuropathy; as well as disorders affecting multiple systems of the body such as systemic lupus erythematosus, systemic vasculitis, polymyositis, Sjögren's syndrome, and myositis. Treating these neurological disorders has proven to be a difficult and elusive task due to the complexity of the disorders and the systems they affect.

Connective tissue disorders are often caused by an autoimmune response of the body, making them difficult to manage. Presently, there is no way of inhibiting the tissue degradation that results from such conditions; instead, physicians have prescribed compounds that act to mask the symptoms, i.e., ameliorate the pain associated with such conditions. However, since there is no way to inhibit the tissue degradation, destruction of the tissues continues unabated despite symptomatic relief. Eventually, the tissues are destroyed by the disease process and dysfunction occurs.

Human skin is a composite material of the epidermis and the dermis. The topmost part of the epidermis is the stratum corneum. This layer is the stiffest layer of the skin, as well as the one most affected by the surrounding environment. Below the stratum corneum is the internal portion of the epidermis. Below the epidermis, the topmost layer of the dermis is the papillary dermis, which is made of relatively loose connective tissues that define the micro-relief of the skin. The reticular dermis, disposed beneath the papillary dermis, is tight, connective tissue that is spatially organized. The reticular dermis is also associated with coarse wrinkles. At the bottom of the dermis lies the subcutaneous layer.

The principal functions of the skin include protection, excretion, secretion, absorption, thermoregulation, pigmentogenesis, accumulation, sensory perception, and regulation of immunological processes. These functions are detrimentally affected by the structural changes in the skin due to aging and excessive sun exposure. The physiological changes associated with skin aging include impairment of the barrier function and decreased turnover of epidermal cells, for example. [Cerimele, D., et al., Br. J. Dermatol., 122 Suppl. 35, p. 13-20 (April 1990)].

The mechanical properties of the skin, such as elasticity, are controlled by the density and geometry of the network of collagen and elastic fiber tissue therein. Damaged collagen and elastin lose their contractile properties, resulting in skin wrinkling and skin surface roughness. As the skin ages or becomes unhealthy, it acquires sags, stretch marks, bumps, bruises or wrinkles, it roughens, and it has reduced ability to synthesize Vitamin D. Aged skin also becomes thinner and has a flattened dermoepidermal interface because of the alterations in collagen, elastin, and glycosaminoglycans. [Fenske, N. A, and Lober, C. W., J. Am. Acad. Dermatol., 15:571-585 (October 1986); Montagna, W. and Carlisle, K., Journal of Investigative Dermatol., 73(1):47-53 (1979)].

Cellulite is a cosmetic/medical condition caused by defects in the skin that result in the skin having an “orange peel” or “cottage cheese” effect. Cellulite is typically characterized by dermal deterioration due to a breakdown in blood vessel integrity and a loss of capillary networks in the dermal and subdermal levels of the skin. The vascular deterioration tends to decrease the dermal metabolism. This decreased metabolism hinders protein synthesis and repair processes, which results in dermal thinning. The condition is further characterized by fat cells becoming engorged with lipids, swelling, and clumping together, as well as excess fluid retention in the dermal and subdermal regions of the skin. Thus, individuals afflicted with cellulite tend to have a thicker subcutaneous fatty layer of skin. In the advanced stages of cellulite, reticular protein deposits called septa begin to form around the fatty deposits in the skin and occlude the fat cells. As the condition further progresses, hard nodules of fat cells and clumps of fat surrounded by septa form in the dermal region. This leads to the surface of the skin displaying considerable heterogeneity and being characterized as having a “cottage cheese” appearance. This appearance is most pronounced in overweight individuals. Individuals with cellulite also tend to have a thinner epidermis and dermis in the affected region, decreased firmness of the skin, and decreased rate of cell renewal.

The appearance of cellulite currently tends to be treated by administering xanthines, which include caffeine, theophylline, and aminophylline. Xanthines acts as a diuretic that removes water from the fat cells and thus reduces the size of the fat cells. The effect of xanthines, however, is temporary and the fat cells become rehydrated as soon as the individual replenishes the lost water.

A variety of vitamins and minerals have individually been administered to treat certain skin and other problems that occur when the patient has a deficiency of that vitamin or mineral. Vitamin A, for example, assists in the treatment of acne and to facilitate wound healing; vitamin C (ascorbic acid) assists in the prevention of skin bruising and wound healing; vitamin E is an antioxidant; and copper assists in the treatment of elastic tissue defects. [Neldner, K. H., Amer. Acad. Derm. Annl. Mtg., Wash D.C., Dec. 6, 1993]. Topical use of vitamin C is also believed to ward off sun damage, reduce breakdown of connective tissues, and possibly promote collagen synthesis. [Dial, W., Medical World News, p. 12, March 1991]. Vitamin E is used topically as an anti-inflammatory agent, for enhancement of skin moisturization, for UV-ray protection of cells, and for retardation of premature skin aging.

Catechin-based preparations, including proanthanols and proanthocyanidins are powerful antioxidants. These compounds are found in flowers, plant leaves, and grape seeds, for example. [Lubell, A., Cosmetic Dermatol., 9(7):58 & 60 (July 1996)].

N-Acetylglucosamine and glucosamine have been examined for use in the prevention and treatment of degenerative joint diseases and cartilage loss, and found to increase the glycosaminoglycans present in the cartilage to restore cartilage. [See Grevenstein, J., et al., Acta Orthopaedia Belgica, 57(2):157-161 (1991); Setnikar, I., Drug Res., 36(4):720-733 (1986); Drovanti, A., et al, Clin. Therap., 3(4):1-6 (1980)]. Glucosamine has also been examined in connection with arthritis [See, e.g., Murray, M. T.] and oral and injected glucosamine have been reported to be useful for arthrosic patients. [Tapadinhas, M. J., et al., Pharmatherapeutica, 3(3):157-168 (1982); D'Ambrosio, E., et al., Pharmatherapeutica, 2(8):504-508 (1981)].

The metabolism of glycosaminoglycans under the influence of herbal and other anti-inflammatory agents has been examined by measuring glycosaminoglycans in the skin, liver, kidney, and spleen after administration of several compounds. [Reddy, G. K., et al., Biochem. Pharmacology, 38(20):3527-3534 (1989)].

In addition to their individual use to supplement a deficiency in a patient, various of the above ingredients have been combined to form pharmaceuticals designed to prevent and treat certain cellular, skin, and other conditions. For example, U.S. Pat. No. 3,773,930 discloses a low residue, dietary composition having at least one amino acid and a quantity of non-amino acid derived caloric material sufficient to obviate the diarrhea problem of straight amino acid compositions. A flavoring material may also be included to render the composition more palatable.

U.S. Pat. No. 4,285,964 discloses a salt of (+)-catechin formed by reacting (+)-catechin with at least a basic amino acid, such as L-lysine and L-arginine; and a hydrosoluble double salt formed from the reaction product of (+)-catechin with a basic amino-acid, such as L-lysine and L-arginine, and another inorganic or organic acid. The patent further discloses methods of treating degenerative diseases of the connective tissue by topically administering the composition.

U.S. Pat. No. 4,414,202 discloses a composition for the treatment of skin wounds with a buffered salt solution having a pH between 6 to 7.8 and administering a starch hydrolysate compound, and preferably including alphaketoglutaric acid or alphaketoglutarate salts. Optional additives to the composition include ascorbic acid or salts thereof, ferrous salts, and glycine, L-Proline, and L-Lysine.

U.S. Pat. No. 4,424,232 discloses a topical composition for the treatment of herpes simplex, cold sores, lesions, and other painful skin conditions including L-lysine, gibberellic acid, and urea in an inert carrier having water. The composition may also include L-ascorbic acid, as well as methyl paraben, propyl paraben, or mixtures thereof.

U.S. Pat. No. 4,647,453 discloses a method and composition for treatment of tissue degenerative inflammatory disease in animals and humans by oral administration of ascorbic acid, bioavailable calcium, a precursor or stimulant of epinephrine or nor-epinephrine of tyrosine or phenylalanine, and an anti-inflammatory substance selected from anti-inflammatory sugars, amino sugars and biocompatible acid addition salts thereof, and anti-inflammatory amino acids, to promote connective tissue regrowth.

U.S. Pat. No. 5,198,465 discloses a composition for treating precursor deficiencies in the synthesis of collagen with proline, glycine, lysine, vitamin C, and one or more compounds selected from α-ketoglutaric acid, methionine, cysteine, cystine, valine, and pharmaceutically acceptable diluents and excipients.

U.S. Pat. Nos. 5,332,579 and 5,308,627 disclose a nutritional supplement to assist persons recovering from addiction by administering a variety of vitamins and minerals including enzyme activating substances such as magnesium and zinc; an enzyme co-factor that is a vitamin like various vitamin B complexes; an, enzyme producer such as an amino acid like glutamic acid; an herbal antispasmodic substance like Valerian root; and vitamin C.

U.S. Pat. No. 5,415,875 discloses a method of suppressing formation of lipid peroxide and removing peroxide by applying to the skin a decomposed product of shell membrane and tocopherol and derivatives. Lysine, proline, Vitamin C, for examples, are listed among a vast genus of optional additives.

The above references, however, do not teach pharmaceutical compositions or methods for managing connective tissue disorders. The present invention provides such a method. The method manages connective tissue disorders by administering to a patient in need of treatment a composition that supplements collagen and elastic tissue in the connective tissue.

Additionally, the above references, however, do not teach pharmaceutical compositions or methods for reducing or eliminating the appearance of cellulite. The present invention provides such a method. The method reduces or eliminates the appearance of cellulite by administering to a patient in need of treatment a composition that supplements collagen and elastic tissue in the skin and thickens the dermis.

### SUMMARY OF THE INVENTION

The present invention relates to methods for managing connective tissue disorders in a patient. The method involves administering to a patient in need of treatment a sugar compound that is converted to a glycosaminoglycan, a primary antioxidant component, at least one amino acid component, at least one transition metal component, at least one moisturizing agent, and at least one fatty acid.

The sugar, the primary antioxidant, the at least one amino acid component, the at least one transition metal component, the at least one moisturizing agent, and the at least one fatty acid may be administered simultaneously in the form of a single pharmaceutical composition. The composition may be administered orally or topically. When the composition is administered orally, it may be administered as a tablet or capsule having 1 mg to 2,000 mg of the composition. When the composition is administered topically to the patient's skin, it is preferably administered with one or more mono- or poly-hydroxy acids or tannic acid, a mixture thereof, or a pharmaceutically acceptable salt or ester thereof in an amount sufficient to exfoliate at least a portion of the skin. The mono- or poly-hydroxy acid may be glycolic acid or salicylic acid. The composition may further be topically administered with hydrogen peroxide in an amount sufficient to cleanse the skin.

In another preferred embodiment, the method further includes administering at least one vascular dilator. Preferred vascular dilators include extract of ginko biloba, ginsing, phenylalanine, niacin or a mixture thereof. When the vascular dilator is extract of ginko biloba, it is preferable to administer from about 5 mg to 300 mg of extract of ginko biloba. When the vascular dilator is ginsing it is preferable to administer from about 100 mg to 200 mg of ginsing extract. When the vascular dilator is phenylalanine it is preferable to administer from about 75 mg to 1500 mg of phenylalanine. When the vascular dilator is niacin, it is preferable to administer from about 5 mg to 1500 mg of niacin.

The sugar compound may be N-acetylglucosamine, or a pharmaceutically acceptable salt thereof, in an amount of about 40 to 1500 mg. The anti-oxidant may be a vitamin C source in an amount of about 35 to 200 mg. In another embodiment, the at least one amino acid component is selected from lysine, proline, cysteine, glycine, methionine, and mixtures thereof in an amount of about 35 to 750 mg. In yet another embodiment, the at least one transition metal component is selected from zinc, manganese, copper, and mixtures thereof. In still another embodiment, the at least one moisturizing agent is a hydrophobic agent, hydrophilic agent, or a combination thereof.

The method for managing connective tissue disorders may further include administering to the person a proanthanol or proanthocyanidin. In another preferred embodiment, the method further includes administering a glucosamine, or a pharmaceutically acceptable salt or ester thereof, and chondroiton. In a more preferred embodiment, the proanthocyanidin is grape seed extract.

In a preferred embodiment, the method further includes administering a fruit extract in an amount of from about 0.1 mg to 2,000 mg. In a more preferred embodiment, the fruit extract is obtained from one or more of apricots, apples, pears, peaches, pineapples, papayas, cherries, kiwis, tangerines, oranges, and pomegranates. In another preferred embodiment, the fatty acid is selected from linolenic acid, alpha-linoleic acid, gamma-linoleic acid, arachidonic acid, or a mixture thereof.

The invention further relates to pharmaceutical compositions for managing connective tissue disorders in a patient. The composition includes a sugar compound that is converted to a glycosaminoglycan, a primary antioxidant component, at least one amino acid component, at least one transition metal component, at least one moisturizing agent, and at least one fatty acid. Additionally, an exfoliant may be further included. The exfoliant can be a mono- or poly-hydroxy acid, a mixture thereof, or a pharmaceutically acceptable salt or ester thereof.

In a preferred embodiment, the composition for topical administration to the patient's skin further included hydrogen peroxide in an amount sufficient to cleanse the skin.

### DETAILED DESCRIPTION OF THE PREFERRED EMBODIMENTS

A method for treating connective tissue disorders has now been discovered. Additionally, a method for reducing or eliminating the appearance of cellulite has also been discovered. The management of these connective tissue disorders and the reduction or elimination of the appearance of cellulite may advantageously be accomplished by the administration of the pharmaceutical compositions of the present invention.

The term “connective tissue”, as used herein, means any of the class of tissues wherein the extracellular components, including fibers and intercellular substances, are abundant. These tissues include, but are not limited to, areolar or loose tissues; adipose tissues; dense, regular, or irregular tissues; white fibrous tissues; elastic tissues; mucous tissues; and lymphoid tissues, as well as other tissue groups that include, but are not limited to, tendons, ligaments, aponeuoses, blood vessels, nerves, vitreous humor, cartilage, scalp, nails, and bone.

The term “connective tissue disorder”, as used herein, means any disorder, ailment, malady, or dysfunction affecting connective tissues often, but not necessarily, caused by an autoimmune process, including, but not limited to, joint inflamation and stiffness, systemic lupus erythematosus, systemic vasculitis, polymyolitis or dermatomyositis, scleroderma, systematic sclerosis, Sjögren's syndrome, arthritis, polyarteritis nodosa, and dermatopolymyositis, myositis, and rheumatoid arthritis.

The terms “managing” or “management,” as used herein, includes one or more of the prevention, treatment, or modification of a connective tissue disorder.

The method includes administering to a human in need of treatment therapeutically sufficient amounts of at least one sugar compound which is converted into glycosaminoglycans in the bloodstream, a primary antioxidant component, at least one amino acid component, at least one transition metal component, at least one moisturizer component, and at least one fatty acid. Without wishing to be bound by theory, Applicant believes that the combination of these materials synergistically strengthens and thickens the collagen and/or elastin in the connective tissue, whereby relief from connective tissue disorders is derived. Improved compositions have also been discovered that are used in preferred methods. These compositions and methods preferably include a vascular dilator and/or hydrogen peroxide.

In one embodiment the method of the invention involves treating a connective tissue disorder associated with aging. Representative connective tissue disorders associated with aging include, but are not limited to, arthritis; wrinkles; decubitus disorders such as bed sores and other skin ulcerations, dermatomyositis, systematic sclerosis, decubitus disorders, scleroderma, thinning skin, age spots, abnormal pigmentation, leathery skin, yellowish discoloration of the skin, dry skin, stretch marks, loss of skin elasticity, and bed sores; neurological disorders such as peripheral neuropathy, Alzheimer disease, senility, peripheral neuropathy; as well as disorders affecting multiple systems of the body such as systemic lupus erythematosus, systemic vasculitis, polymyositis, Sjögren's syndrome, and myositis; and circulatory disorders such as poor circulation and varicose veins, arthritis, dilated blood vessels, and polyarthritis nodosa. Aging is accompanied with a decrease in both the amount of the functioning of connective tissue. Without wishing to be bound by theory it is believed that the methods of the invention treat connective tissue disorder associated with aging by strengthening the connective tissue.

In another embodiment the method involves treating a neurological disorder. Neurological disorders include, but are not limited to, Alzheimer disease, peripheral neuropathy, senility, dementia, as well as disorders affecting multiple systems of the body such as systemic lupus erythematosus, systemic vasculitis, polymyositis, Sjögren's syndrome, and myositis. Without wishing to be bound by theory it is believed that the methods of the invention treat neurological disorders by strengthening nerve tissue.

A method for reducing or eliminating the appearance of cellulite has also been discovered. The method includes administering to a human in need of treatment therapeutically sufficient amounts of at least one sugar compound which is converted into glycosaminoglycans in the bloodstream, a primary antioxidant component, at least one amino acid component, and at least one transition metal component, so as to modify the thickness of the skin to reduce or eliminate the appearance of cellulite. Without wishing to be bound by theory, Applicant believes that a thicker dermis desirably reduces the appearance of cellulite that occurs when areas of the skin become thin. Improved compositions have also been discovered that are used in preferred methods. These compositions and methods preferably include a vascular dilator and/or a fat burner.

In another embodiment, the compositions and methods of the present invention improve hair growth and reduce loss of hair. Without wishing to be bound by theory, it is believed that the compositions of the invention when applied to the scalp strengthen the blood vessels and skin of the scalp. By strengthening the connective tissue around the hair follicles, hair growth is improved, and hair loss is reduced.

The composition preferably contains at least one sugar compound, and more preferably just one sugar compound, present in about 5 to 50 weight percent, preferably about 10 to 40 weight percent, and more preferably about 15 to 30 weight percent of the composition. The primary antioxidant component is preferably present in an amount of about to 50 weight percent, more preferably about 10 to 40 weight percent, and most preferably about 15 to 30 weight percent of the composition. The amino acid component is preferably present in about 8 to 60 weight percent, more preferably about 15 to 50 weight percent, most preferably about 20 to 40 weight percent of the composition. The transition metal component is preferably present in about 0.5 to 15 weight percent, more preferably about 2 to 12 weight percent, and most preferably present about 5 to 10 weight percent of the composition. The moisturizer component is preferably present in about 0.01 to 20 weight percent, preferably about 0.05 to 10 percent, more preferably about 0.1 to 5 weight percent of the composition. The fatty acid is preferably present in about 0.01 to 20 weight percent, preferably about 0.05 to 15 weight percent, more preferably from about 0.1 to 5 weight percent of the composition.

The first component of the composition is any sugar compound that is converted to a glycosaminoglycan in the human bloodstream. Typically, this would be an N-acetylglucosamine compound, or a pharmaceutically acceptable salt or ester thereof. The N-acetylglucosamine component may be N-acetylglucosamine or any pharmaceutically acceptable salt or ester thereof, but more preferably is the N-acetylglucosamine only. This component must be present in sufficient quantity in the pharmaceutical composition to promote thickening of the dermis and connective tissue. Without wishing to be bound by theory it is believed that one mechanism by which glycosaminoglycans help thicken the dermis and connective tissue is by improving the ability of the dermis and connective tissue to absorb moisture. It is also believed that glycosaminoglycans are an important factor in assisting fibroblasts in producing collagen and elastic tissue. Thus, it can be advantageous to administer the composition in conjunction with a topically administered exfoliant that further improves the skin's ability to absorb moisture by removing dead and dying skin cells from at least a portion of the skin. The N-acetylglucosamine is present in about 5 to 30 weight percent, preferably 8 to 27 weight percent, and more preferably 12 to 24 weight percent of the pharmaceutical composition. A unit dose of N-acetylglucosamine is typically about 40 mg to 1500 mg, preferably about 60 to 750, and more preferably about 100 mg to 200 mg.

The pharmaceutical composition includes a primary antioxidant component. Any antioxidant known to those skilled in the art can be used in the methods and compositions of the invention. Suitable antioxidants for use in the methods and compositions of the invention include, but are not limited to, vitamin C sources, preferably ascorbic acid, or a pharmaceutically acceptable salt or ester thereof, and more preferably ascorbyl palmitate, dipalmitate L-ascorbate, sodium L-ascorbate-2-sulfate, glucosamine ascorbate, or an ascorbic salt, such as sodium, potassium, or calcium ascorbate, or mixtures thereof. The antioxidant component inhibits collagenase and elastase, enzymes that break down collagen and elastic tissues. In addition, vitamin C also strengthens blood vessels. When oral formulations of the pharmaceutical composition are used, it is preferred that a non-acidic form of vitamin C be used to reduce the stomach irritation that may occur when using an acidic form. The vitamin C source is present in the pharmaceutical composition in about 5 to 50 weight percent, preferably about 7 to 40 weight percent, and more preferably about 10 to 25 weight percent. A unit dose when a vitamin C source is the primary antioxidant component is typically from about 40 mg to 400 mg, preferably about 60 mg to 300 mg, and more preferably about 80 mg to 150 mg. Vitamin C is also approved by the FDA and has wide consumer acceptance, so that it can be used in amounts as high as 10,000 mg, if desired.

The pharmaceutical composition also includes at least one amino acid to assist in thickening and strengthening the skin and connective tissue. The amino acids assist in the thickening of the skin and connective tissue and supplementing of collagen and elastic tissues. In one embodiment, the amino acid is an essential amino acid. Essential amino acids include, but are not limited to, lysine, leucine, isoleucine, methionine, phenylalanine, threonine, tryptophan, histidine, taurine, cysteine, and valine. Preferably two or more amino acids are used in combination. Either the L- or D- forms of amino acids are acceptable. Lysine, proline, and glycine are the most preferred amino acids and they are advantageously used in combination. Cysteine, glycine, methionine or other amino acids can also be used, if desired. The amino acid(s) may be included in a soluble form such as the hydrochloride, i.e., L-Lysine hydrochloride. The amino acid(s) are present in an amount of about 2 to 25 weight percent each, preferably about 4 to 20 weight percent each, and more preferably about 6 to 15 weight percent each. A unit dose for each amino acid is typically about 35 mg to 750 mg each, preferably about 50 mg to 150 mg each, and more preferably about 70 mg to 120 mg in the pharmaceutical composition. Additional useful forms of amino acid include the following: a cysteine source, preferably N-acetyl cysteine, can be present in an amount of about 1 to 10 weight percent, preferably about 2 to 8 weight percent, and more preferably about 3 to 6 weight percent of the pharmaceutical composition. A methionine source, preferably L-selenomethionine, can be present in an amount of about 0.1 to 5 weight percent, preferably 0.2 to 3 weight percent, and more preferably 0.3 to 1 weight percent of the composition, wherein the selenium component is from about 0.1 to 3 weight percent of the methionine source.

One or more transition metal compounds are included in an amount effective to bind collagen and elastic tissue to rebuild the skin and connective tissue. Certain transition metal compounds also inhibit elastase, an enzyme that also breaks down collagen and elastic tissue. Preferred transition metals include zinc, manganese and copper, with combinations thereof being most preferred.

A zinc component can be included to assist in binding collagen and elastic fibers, which both assist in the rebuilding of the skin and connective tissue. The zinc component may be any zinc compound or pharmaceutically acceptable salt thereof, but more preferably is a zinc complexed with an amino acid, and most preferably is zinc monomethionine, wherein the zinc is typically present in about 10 to 30 weight percent of the complex. The zinc component is present in about 1 to 10 weight percent, more preferably about 2 to 7 weight percent, and most preferably about 3 to 5 weight percent of the pharmaceutical composition.

A manganese component can also be included to assist in binding collagen and elastic fibers and to increase cellular energy. The manganese component may be any manganese compound or pharmaceutically acceptable salt thereof, but more preferably is a manganese component which is at least partially complexed with a vitamin C source, and most preferably is manganese ascorbate or manganese ascorbic acid, wherein the manganese is typically present in about 5 to 20 weight percent of the complex. When complexed with vitamin C, this vitamin C source may be included in the overall percentage of vitamin C in the pharmaceutical composition. The manganese component is typically present in about 1 to 10 weight percent, more preferably about 2 to 7 weight percent, and most preferably about 2.5 to 4 weight percent of the pharmaceutical composition.

A copper component is preferably also included in the pharmaceutical composition, and may be any copper compound or pharmaceutically acceptable salt thereof, but preferably is copper sebacate, wherein the copper is typically present in about 5 to 20 weight percent of the copper sebacate. The copper component also inhibits elastase and is present in about 0.1 to 5 weight percent, preferably about 0.2 to 3 weight percent, and more preferably about 0.3 to 1 weight percent of the pharmaceutical composition. A unit dose of the copper component of the pharmaceutical composition may include about 1 mg to 40 mg, preferably about 2 mg to 25 mg, and more preferably about 2.5 mg to 10 mg of the pharmaceutical composition.

One or more moisturizing agents are included in an amount to hydrate the skin. “Moisturizing agent,” as used herein, is used to include any agent that facilitates hydration of the skin by inhibiting or preventing loss of water from the skin; absorbs water from the atmosphere and hydrates the skin; or enhances the ability of the skin to absorb water directly from the atmosphere; or a combination thereof. Without wishing to be bound by theory it is believed that the moisturizing agent further improves the skin's ability to absorb the pharmaceutical compositions. It is also believed that the moisturizing agent helps thicken the skin and connective tissue by improving the ability of the skin and connective tissue to absorb moisture. Maintaining moisture within the connective tissues provides improved function of the connective tissues and enhanced efficacy in managing connective tissue disorders and reducing or eliminating the appearance of cellulite. Suitable moisturizing agents include, but are not limited to, hydrophobic agents, and hydrophilic agents, or combinations thereof. Moisturizers, when used, are typically present in an amount from about 0.01 to 20 weight percent, preferably about 0.05 to 10 weight percent, more preferably from about 0.1 to 5 weight percent of the composition.

Moisturizing agents that are hydrophobic agents include, but are not limited to, ceramide, borage oil, tocopherol (Vitamin E), tocopherol linoleate, dimethicone, glycerine, and mixtures thereof. Hydrophobic agents, when present, are believed to moisturize the skin by inhibiting or preventing the loss of water from the skin. The hydrophobic agent, when present, is typically present in an amount from about 0.01 to 20 weight percent, preferably from about 0.05 to 15 weight percent, and more preferably from about 0.1 to 5 weight percent of the composition.

Moisturizing agents that are hydrophilic agents include, but are not limited to, hyaluronic acid, sodium peroxylinecarbolic acid (sodium PCA), wheat protein (e.g., laurdimonium hydroxypropyl hydrolyzed wheat protein), hair keratin amino acids, and mixtures thereof. Sodium chloride may also be present, particularly when hair keratin amino acids are included as a moisturizer. Hydrophilic agents, when present, are believed to moisturize the skin by absorbing moisture from the atmosphere to hydrate or facilitate hydration of the skin. The hydrophilic agent, when present, is typically present in an amount from about 0.01 to 20 weight percent, preferably from about 0.05 to 15 weight percent, and more preferably from about 0.1 to 5 weight percent of the composition.

Other moisturizing agents that hydrate the skin and connective tissue and are useful in the compositions and methods of the present invention include, but are not limited to, panthenol; primrose oil; GLA 3 and other fish oils that may include, for example, the omega-3 and omega-6 oils; and flax seed oil. Preferably, these moisturizing agents are administered orally.

In a preferred embodiment, the pharmaceutical compositions of the invention include both hydrophobic and hydrophillic agents. Without wishing to be bound by theory it is believed that the hydrophobic agent prevents the loss of water from the skin while the hydrophillic agents absorb moisture from the atmosphere to hydrate or facilitate hydration of the skin. The synergistic effect provides enhanced efficacy in managing connective tissue disorders and reducing or eliminating the appearance of cellulite.

The pharmaceutical compositions of the invention include at least one fatty acid. Any fatty acid known to those skilled in the art can be used in the methods and compositions of the invention. Suitable fatty acids for use in the methods and compositions of the invention include, but are not limited to, arachidonic acid, linolenic acid, alpha-linoleic acid, gamma-linoleic acid, dihomogamma-linolenic acid, stearidonic acid, eicosapentaenoic acid, docosahexaenoic acid, or combinations thereof. The fatty acid is typically present in an amount from about 0.01 to 20 weight percent, preferably from about 0.05 to 15 weight percent, and more preferably from about 0.1 to 5 weight percent of the composition. Without wishing to be bound by theory, it is believed that the fatty acid acts as an anti-inflammatory, and also increases the stability and strength of the cell membranes in connective tissues.

Without wishing to be bound by theory it is believed that the fatty acid, in combination with the other components of the composition, interacts in a synergistic manner to strengthen connective tissues. The fatty acid acts in combination with the other components to replace lipid structures of the cell membrane, thereby strengthening the connective tissues. The synergistic effect provides enhanced efficacy in managing connective tissue disorders and reducing or eliminating the appearance of cellulite.

A preferred embodiment of the invention further includes hydrogen peroxide in an amount sufficient to cleanse the skin. The hydrogen peroxide is present in an amount sufficient to cleanse at least a portion of the skin. “Cleanse” as used herein includes the removal of dirt, debris, air pollutants, desquamating cells, and cutaneous secretions of the dermatological surface. Preferably, the hydrogen peroxide is present in an amount to cleanse the skin without substantial irritation. The hydrogen peroxide is typically present in an amount from about 0.01 to 6 weight percent, preferably 0.05 to 4 weight percent, and more preferably 0.1 to 1 weight percent of the composition. Without wishing to be bound by theory it is believed that cleansing the skin with hydrogen peroxide improves penetration of the pharmaceutical compositions into the skin and, thus, improves the efficacy of the pharmaceutical compositions. Compositions that contain hydrogen peroxide are typically topical compositions.

Without wishing to be bound by theory it is believed that the hydrogen peroxide and the moisturizing agent of the invention interact in a synergistic manner to aid the delivery of the pharmaceutical composition. The hydrogen peroxide cleanses the skin, removes substances foreign to the skin, and improves penetration of the pharmaceutical compositions into the skin. The moisturizing agent moisturizes the skin and further improves penetration of the pharmaceutical compositions into the skin. The synergistic effect provides enhanced efficacy in managing connective tissue disorders and reducing or eliminating the appearance of cellulite.

In another preferred form of the invention, the pharmaceutical composition further includes a catechin-based preparation, such as a proanthanol or proanthocyanidin, along with glucosamine or a pharmaceutically acceptable salt or ester thereof, and chondroitin or a pharmaceutically acceptable salt or ester thereof.

The catechin-based preparation, similar to vitamin C, inhibits elastase and collagenase, which is another enzyme that attacks elastic tissue and collagen. The catechin-based preparation is preferably a proanthanol or proanthocyanidin, more preferably a proanthocyanidin, and most preferably grape seed extract. These compounds are considered to be secondary antioxidants, because they are present in lesser amounts than the primary antioxidant. The catechin-based preparation is present in about 0.5 to 5 weight percent, more preferably about 0.6 to 3 weight percent, and most preferably about 0.7 to 2 weight percent of the pharmaceutical composition.

The glucosamine or a pharmaceutically acceptable salt or ester thereof, and the chondroitin or a pharmaceutically acceptable salt or ester thereof, are each present in about 3 to 17 weight percent, preferably about 4 to 12 weight percent each, and more preferably about 5 to 8 weight percent each of the pharmaceutical composition. The glucosamine component preferably is present as a sulfate or succinate, and more preferably is D-glucosamine sulfate, wherein the glucosamine is preferably present as about 60 to 90 weight percent of the salt. The glucosamine content of this component contributes to the formation of glycosoaminoglycans in the connective tissues. The chondroitin component preferably is present as a sulfate or succinate, and more preferably is chondroitin sulfate, wherein the chondroitin is preferably present as about 65 to 95 weight percent of the salt.

In another preferred embodiment, the pharmaceutical composition includes at least one fruit extract, which provides antioxidants that are naturally present in the fruit extracts. Preferably, the fruit extract is obtained from apricots, apples, pears, peaches, pineapples, papayas, cherries, kiwis, tangerines, or oranges. Most preferably, the fruit extract is obtained from pomegranate. The fruit extract is preferably present in an amount of about 0.01 to 80 weight percent and more preferably in about 0.1 to 20 weight percent of the pharmaceutical composition. A preferred oral daily dose range of the fruit extract, when included in the composition, should be from about 0.01 mg to 2,000 mg; more preferably about 400 mg to 1,600 mg; and most preferably about 800 mg to 1,200 mg. In general, a preferred topical daily dosage range, in single or divided doses, should be from about 0.01 mg to 20,000 mg, more preferably about 2,000 mg to 16,000 mg and most preferably 6,000 mg to 10,000 mg of the fruit extract.

Pharmaceutical compositions adapted for topical administration can further comprise an exfoliant. The exfoliant can be an enzymatic exfoliant, such as papain or bromalein, or a mono- or poly-hydroxy acid, a mixture thereof, or a pharmaceutically acceptable salt or ester thereof. Any enzymatic exfoliant, or mono- or poly-hydroxy acid known to those skilled in the art may be used in the compositions and methods of the invention. Examples of enzymatic exfoliants include, but are not limited to, papain, from papaya, and bromalein, from pineapple. Examples of suitable mono- or poly-hydroxy acids include, but are not limited to, alkyl hydroxycarboxylic acids, aralkyl and aryl hydroxycarboxylic acids, polyhydroxy-carboxylic acids, and hydroxy-polycarboxylic acids. Exemplary mono- or poly-hydroxy acids include: 2-hydroxyacetic acid (glycolic acid); 2-hydroxypropanoic acid (lactic acid); 2-methyl 2-hydroxypropanoic acid; 2-hydroxybutanoic acid; phenyl 2-hydroxyacetic acid; phenyl 2-methyl 2-hydroxyacetic acid; 3-phenyl 2-hydroxyacetic acid; 2,3-dihydroxypropanoic acid; 2,3,4-trihydroxybutanoic acid; 2,3,4,5,6-pentahydroxyhexanoic acid; 2-hydroxydodecanoic acid; 2,3,4,5-tetrahydroxypentanoic acid; 2,3,4,5,6,7-hexahydroxyheptanoic acid; diphenyl 2-hydroxyacetic acid; 4-hydroxymandelic acid; 4-chloromandelic acid; 3-hydroxybutanoic acid; 4-hydroxybutanoic acid; 2-hydroxyhexanoic acid; 5-hydroxydodecanoic acid; 12-hydroxydodecanoic acid; 10-hydroxydecanoic acid; 16-hydroxyhexadecanoic acid; 2-hydroxy-3-methylbutanoic acid; 2-hydroxy-4-methylpentanoic acid; 3-hydroxy-4-methoxymandelic acid; 4-hydroxy-3-methoxymandelic acid; 2-hydroxy-2-methylbutanoic acid; 3-(2-hydroxyphenyl) lactic acid; 3-(4-hydroxyphenyl) lactic acid; hexahydromandelic acid; 3-hydroxy-3-methylpentanoic acid; 4-hydroxydecanoic acid; 5-hydroxydecanoic acid; aleuritic acid; 2-hydroxypropanedioic acid; 2-hydroxybutanedioic acid; erythraric acid; threaric acid; arabiraric acid; ribaric acid; xylaric acid; lyxaric acid; glucaric acid; galactaric acid; mannaric acid; gularic acid; allaric acid; altraric acid; idaric acid; talaric acid; 2-hydroxy-2-methylbutanedioic acid; citric acid; isocitric acid; agaricic acid; quinic acid; glucoronic acid; glucoronolactone; galactoronic acid; galactoronolactone; uronic acids; uronolactones; ascorbic acid; dihydroascorbic acid; dihydroxytartaric acid; tropic acid; ribonolactone; gluconolactone; galactonolactone; gulonolactone; mannonolactone; citramalic acid; pyruvic acid; hydroxypyruvic acid; hydroxypyruvic acid phosphate and esters thereof; methyl pyruvate; ethyl pyruvate; propyl pyruvate; isopropyl pyruvate; phenyl pyruvic acid and esters thereof; methyl phenyl pyruvate, ethyl phenyl pyruvate, and propyl phenyl pyruvate; formyl formic acid and esters thereof; methyl formyl formate, ethyl formyl formate, and propyl formyl formate; benzoyl formic acid and esters thereof; methyl benzoyl formate, ethyl benzoyl formate, and propyl benzoyl formate; 4-hydroxybenzoyl formic acid and esters thereof; 4-hydroxyphenyl pyruvic acid and esters thereof; 2-hydroxyphenyl pyruvic acid and esters thereof; and mixtures thereof. The hydroxy acids are preferably selected from one or more alpha-hydroxy acids or beta-hydroxy acids, more preferably from glycolic, lactic, citric, tannic, or salicylic acid, and most preferably from citric or salicylic acids. It should be understood that one or more derivatives of the above acidic component, such as esters or lactones thereof, are also suitably used. One of ordinary skill in the art will also understand that various hydroxy acids described in U.S. Pat. Nos. 5,547,988 and 5,422,370, which are incorporated herein by express reference thereto, are also suitable for use in the compositions and methods of the invention. The enzymatic and/or acidic components are administered topically in conjunction with the compositions and methods in an amount sufficient to exfoliate, i.e., remove dead or dying skin cells, from at least a portion of the skin. Without wishing to be bound by theory, the exfoliation of the dead or dying skin cells improves the absorption of the pharmaceutical compositions. The enzymatic and/or acidic components, when used, are typically present in an amount from about 0.1 to 12 weight percent, preferably from about 1 to 11 weight percent, more preferably from about 4 to 10 weight percent of the composition. For example, the acidic component may be from about 0.1 to 3 weight percent citric acid in combination with up to about 2 weight percent salicylic acid.

In a more preferred form, several optional additives are included in the pharmaceutical composition, such as a vitamin E source, a vitamin B₃ source, quercetin powder, pyridoxal 5 phosphate-Co B₆, and a vitamin A source. The vitamin E preferably is a sulfate or succinate vitamin E complex, and more preferably is D-alpha tocopheryl acid succinate. The vitamin E source is present in about 1 to 15 weight percent, preferably about 2 to 12 weight percent, and more preferably about 3 to 10 weight percent of the composition. In any event, no more than 1,500 IU should be ingested per day, as Vitamin E becomes toxic at higher doses. The vitamin B₃ source preferably is niacinamide, and the source is present in about 0.5 to 15 weight percent, preferably about 1 to 12 weight percent, and more preferably about 1.5 to 10 weight percent of the composition. The vitamin A source preferably is vitamin A palmitate, and the source is present in about 0.1 to 5 weight percent, preferably 0.2 to 3 weight percent, and more preferably 0.3 to 1 weight percent of the composition. Vitamin A is toxic at high levels, such that no more than 400,000 IU should be cumulatively ingested per day for greater than six months. The quercetin powder is quercetin dihydrate, which is typically present in about 0.5 to 15 weight percent, preferably about 1 to 12 weight percent, and more preferably about 1.5 to 10 weight percent of the composition. The pyridoxal 5 phosphate-Co B₆, also known as P-5-P monohydrate, is typically present in about 0.1 to 5 weight percent, preferably 0.2 to 3 weight percent, and more preferably 0.3 to 1 weight percent of the composition.

In another preferred form, the pharmaceutical composition may also include at least one vascular dilator or “fat burner.” The vascular dilator may be administered in an amount sufficient to improve blood supply to the skin and connective tissue. Without wishing to be bound by theory, vascular dilators are also believed to strengthen blood vessels. Any suitable compound available to one of ordinary skill in the art that improves blood supply may be used. Vascular dilators include, but are not limited to, ginko biloba, ginsing, phenylalanine, and mixtures thereof. When the vascular dilator is ginsing it is preferable to administer from about 100 mg to 200 mg per day of a standardized herbal extract of ginsing that supplies approximately 4-7% ginsenosides, the active ingredients in ginsing. When the vascular dilator is phenylalanine it is preferable to administer from about 75 mg to 1500 mg of phenylalanine per day. The typical dose for phenylalanine is 200 mg administered three times per day. The preferred vascular dilator is extract of ginko biloba. Extract of ginko biloba contains as the active ingredient the vascular dilator ginkoflavone glycoside. A unit dose of ginko biloba is typically about 5 mg to 300 mg, preferably about 20 mg to 200 mg, and more preferably about 40 mg to 160 mg. The most preferred form of the vascular dilator is two tablets per day each containing about 60 mg of ginko biloba extract. A sufficient amount of vascular dilator can readily be determined by one of ordinary skill in the art.

“Fat burners” are compounds that reduce absorption of fat in the digestive tract, preferably also the digestion of fat and prevents or inhibits the production of fat. Any suitable compound that reduces absorption or digestion of fat in the digestive tract, or prevents or inhibits the production of fat may be used. Preferred “fat burners” include hydroxy citric acid and chitin. Hydroxy citric acid is believed to prevent or inhibit carbohydrates from being converted into fat. A unit dose of hydroxy citric acid is up to about 3000 mg, preferably from about 500 mg to 2500 mg, more preferably from about 750 mg to 1500 mg. The preferred source of hydroxy citric acid is extract of garcinia cambogia. Thus, when a “fat burner” is included in the compositions or methods of the invention, it is preferred to administer about 1500 mg to 3000 mg of extract of garcinia cambogia, which typically contains about 50 percent hydroxy citric acid. Chitin interferes with the absorption of fat in the intestinal tract by binding with fat molecules to form large masses that the body cannot absorb and are excreted. Chitin can be administered in any amount, but it is typically administered in an amount from about 1000 mg to 2000 mg. A sufficient amount of fat burner can readily be determined by one of ordinary skill in the art.

Optionally, the composition further includes chromium picolinate. Chromium picolinate facilitates entry of sugar into cells and thus enhances the body's ability to utilize nutrients and its own energy resources. The more efficient utilization of energy resources includes more efficient metabolism of fats by the body. A typical unit dose for chromium picolinate, when included, is from about 10 mg to 500 mg, preferably from about 100 mg to 350 mg, and more preferably from about 150 mg to 250 mg.

The pharmaceutical compositions of the invention may also include one or more of a local analgesic or anesthetic, antiyeast agent, antiperspirant, antipsoriatic agent, antiaging agent, antiwrinkle agent, sun screen and/or sun blocking agent, skin lightening agent, depigmenting agent, vitamin, hormone, retinoid or immuno-modulators.

The compositions of the invention may further include one or more surfactants, stabilizers, preservatives, coloring agents, water, buffering agents, emulsifying agents, thickeners, solvents, perfuming agents, and the like. Preferably, the water is deionized water. It should be understood that water includes the remainder of a given composition after other ingredients are determined. Although any pharmaceutically acceptable surfactant, stabilizer, preservative, coloring agent, buffering agents, emulsifying agents, thickeners, solvents, or perfuming agents may be used, certain compounds or mixtures are preferred as discussed below.

Preferred surfactants, including both the foaming and non-foaming type, including, but not limited to, sodium laureth sulfate, sodium laureth- 13 carboxylate, disodium laureth sulfosuccinate, disodium cocoamphodiacetate, and the like, and mixtures thereof. More preferably, at least one amphoteric surfactant is included in the composition, such as disodium cocoamphodiacetate. The amphoteric surfactant, in combination with citric acid, inhibits hydrogen peroxide decomposition. The surfactant component may be present in an amount from about 10 to 90 weight percent, preferably about 20 to 80, and more preferably about 30 to 70 weight percent of the composition.

The term “inhibit hydrogen peroxide decomposition,” as used herein, means to at least stop the rate of decomposition from increasing, preferably to inhibit the decomposition entirely, and more preferably to substantially inhibit the decomposition altogether. “Substantially inhibit,” as used herein, means that less than about 10 weight percent, preferably less than about 3 weight percent, and more preferably less than about 1 weight percent, of the hydrogen peroxide decomposes over a three month period of time at 40° C.

A preferred stabilizer includes glycol stearate or PEG-150 distearate. The stabilizer, when used, is typically present in an amount from about 0.1 to 5 weight percent of the composition.

Preferred preservatives include tetrasodium ethylene-diamine tetraacetic acid (EDTA), methylparaben, benzophenone-4, methylchloroisothiazolinone, methylisothiazolinone, and the like, and mixtures thereof. Preservatives, when used, are typically present in an amount from about 0.01 to 6 weight percent, preferably about 0.05 to 4 weight percent, and more preferably from about 0.1 to 2 weight percent.

Preferred coloring agents include FD&C Green No. 3, FD&C Violet No. 2, FD&C Yellow No. 5, FD&C Red No. 40, and the like, and mixtures thereof. The coloring agents, when used, are typically present in an amount from about 0.001 to 0.1 weight percent, and preferably from about 0.005 to 0.05 weight percent of the composition.

The pharmaceutical compositions of the invention may also include a pharmaceutically acceptable carrier. Any suitable pharmaceutically acceptable carrier readily apparent to those of ordinary skill in the art may be combined with the at least one sugar compound, a primary antioxidant component, at least one amino acid component, at least one transition metal component, and at least one moisturizer component, to provide the pharmaceutical compositions of the invention. Pharmaceutically acceptable carriers include, but are not limited to, hydroxypropyl cellulose, starch (corn, potato, rice, wheat), pregelatinized starch, gelatin, sucrose, acacia, alginic acid, sodium alginate, guar gum, ethyl cellulose, carboxymethylcellulose sodium, carboxymethylcellulose calcium, polyvinylpyrrolidone, methylcellulose, hydroxyproply methylcellulose, microcrystalline cellulose, polyethylene glycol, powdered cellulose, glucose, croscarmellose sodium, crospovidone, polacrilin potassium, sodium starch glycolate, tragacanth, calcium carbonate, dibasic calcium phosphate, tribasic calcium phosphate, kaolin, mannitol, talc, cellulose acetate phthalate, polyethylene phthalate, shellac, titanium dioxide, carnauba wax, microcrystalline wax, calcium stearate, magnesium stearate, castor oil, mineral oil, light mineral oil, glycerin, sorbitol, mannitol, stearic acid, sodium lauryl sulfate, hydrogenated vegetable oil (e.g., peanut, cottonseed, sunflower, sesame, olive, corn, soybean), zinc stearate, ethyl oleate, ethyl laurate, agar, calcium silicate, magnesium silicate, silicon dioxide, colloidal silicon dioxide, calcium chloride, calcium sulfate, silica gel, castor oil, diethyl phthalate, glyercin, mono- and di-acetylated monoglycerides, propylene glycol, triacetin, alamic acid, aluminum monostearate, bentonite, bentonite magma, carbomer 934, carboxymethylcellulose sodium 12, carrageenan, hydroxyethyl cellulose, magnesium aluminum silicate, pectin, polyvinyl alcohol, povidine, sodium alginate, tragacanth, xanthan gum, and silicones. For example, preferred topical formulations of the pharmaceutical composition may include a silicon-containing carrier, but in amounts insufficient to cause substantial irritation. Suitable silicones include cyclomethicone or a mixture of cyclopentasiloxane and dimethicone/vinyldimethicone crosspolymer.

The methods and compositions of the present invention can also optionally include a pharmaceutically acceptable antimicrobial agent. Any pharmaceutically acceptable antimicrobial agent available to those of ordinary skill in the art may be used, but preferably at least one of an antibacterial agent, antifungal agent, antiviral agent, or anthelmintic will be used according to the invention. A single broad spectrum antimicrobial agent, i.e., one that is believed to have at least two of antibacterial, antifungal, and antiviral efficacy, including, but not limited to echinacea, golden seal, benzalkonium chloride, benzethonium chloride, iodine, grape seed extract, pomegranate extract, green tea extract or polyphenols, or combinations thereof, may be included. Another suitable antimicrobial agent includes the class of anthelmintics, such as metronidazole, to facilitate treatment of, e.g., tricomona infection. Preferred antiviral agents include, but are not limited to, acyclovir, tamivir, penciclovir, and the like, and mixtures thereof. Preferred antibacterial agents include, but are not limited to, triclosan, neomycin, polymyxin, bacitracin, clindamycin, benzoyl peroxide, a tetracycline, a sulfa drug, a penicillin, a quinolone, a cephalosporin, and mixtures thereof. Preferred antifungal agents include, but are not limited to, farnesol, econazole, fluconazole, clotrimazole, ketoconazole, calcium or zinc undecylenate, undecylenic acid, butenafine hydrochloride, ciclopirox olaimine, miconazole nitrate, nystatin, sulconazole, terbinafine hydrochloride, and the like, and mixtures thereof. Exemplary tetracyclines include doxycycline and minocycline. An exemplary sulfa drug is sulfacetamide. An exemplary cephalosporin is cephalexin (commercially available as KEFLEX). Exemplary quinolones include the floxacins, such as loemfloxacin, ofloxacin, and trovafloxacin. It should be readily understood that any salts, isomers, pro-drugs, metabolites, or other derivatives of these antimicrobial agents may also be included as the antimicrobial agent in accordance with the invention. The antimicrobial agent is typically present in an amount from about 0.01 to 1.5 weight percent, preferably from about 0.1 to 1.2 weight percent, and more preferably from about 0.3 to 1 weight percent of the composition. The antimicrobial agent inhibits the formation of, and may further reduce the presence of, microbes that can cause connective tissue disorders.

Additionally, an immuno-modulators component may be further used in the methods and compositions of this invention in an amount sufficient to boost the immune system to facilitate repair of connective tissues. Suitable immuno-modulators useful in the compositions of the invention include, but are not limited to, interferon, Aldara (Immiquimod), resiquimod, and tacrolimus (Prograf). The immuno-modulators may be present in an amount from about 0.1 to 10 weight percent, preferably from about 0.5 to 5 weight percent of the composition.

The invention also contemplates using stem-cell therapy to improve the function of the connective tissue. In one embodiment of the invention, stem-cells, preferably from the patients own body, are contacted with the patient's connective tissues, typically by injection. The stem cells then develop into new connective tissues and, thus, improve the connective tissues' ability to function.

In another embodiment, the methods and compositions of the present invention can include an anti-inflammatory agent. The anti-inflamatory agent can include non-steroidal agents, steroidal agents, or combinations thereof.

Examples of suitable steroidal anti-inflammatory agents for use in the compositions and methods of the invention include the corticosteroids such as, but not limited to, hydrocortisone, fluocinolone acetonide, halcinonide, halobetasol propionate, clobetasol propionate, betamethasone dipropionate, betamethasone valerate, and triamcinolone acetonide.

Examples of suitable non-steroidal anti-inflammatory agents for use in the compositions and methods of the invention include, but are not limited to, aspirin, ibuprofen, ketoprofen, and naproxen. These anti-inflammatory agents are preferably administered orally. Other non-steroidal anti-inflammatory agents useful in the compositions of the invention include, but are not limited to, aloe vera gel, aloe vera, licorice extract, pilewort, Canadian willow root, zinc, and allantoin. Allantoin is a preferred non-steroidal anti-inflammatory agent. The anti-inflammatory agents are used in an amount sufficient to inhibit or reduce inflammation, preferably in an amount from about 0.02 to 2 weight percent, preferably from about 0.1 to 1.5 weight percent, and more preferably from about 0.2 to 1 weight percent of the composition.

It should be understood, with reference to managing connective tissue disorders, that the anti-inflammatory agents facilitate inhibition or suppression of inflammation any where on the connective tissues. Arnica Montana (a healing herb) and vitamin K can also be used as the anti-inflammatory. Arnica Montana facilitates the healing of the connective tissues and acts as an antiseptic and local anti-inflammatory, and, when used, is typically present in an amount from about 0.1 to 2 weight percent, preferably about 0.2 to 1 weight percent. Vitamin K inhibits or suppresses inflammation and bruising (i.e., acts as an anti-inflammatory and anti-bruising agent) and, when used, is typically present in an amount from about 0.01 to 1 weight percent, preferably from about 0.1 to 0.5 weight percent.

In another embodiment, the methods and compositions of this invention can include at least one of lecithin, inositol, choline, or phosphatidyl choline. These agents are useful to treat both the cellular and non-cellular components of connective tissues. They are particularly effective for strengthening cell membranes.

The phrase “therapeutically effective amount” means that amount of active ingredient(s), e.g., in a pharmaceutical composition, that provides a therapeutic benefit in the treatment, prevention, or management of connective tissue disorders.

The magnitude of a prophylactic or therapeutic dose of the composition in the management of connective tissue disorders will vary with the severity of the condition to be treated and the route of administration. The dose, and perhaps the dose frequency, will also vary according to the age, body weight, and response of the individual patient. In general, the total daily dose range, for the conditions described herein, is from about 10 mg to about 20,000 mg administered in single or divided doses orally, topically, transdermally, or by inhalation. For example, a typical oral daily dose range should be from about 10 mg to 20,000 mg, preferably about 2,000 mg to 16,000 mg, and more preferably about 6,000 mg to 10,000 mg of the active components (i.e., excluding excipients and carriers).

It is further recommended that children, patients aged over 65 years, and those with impaired renal or hepatic function initially receive low doses, and that they then be titrated based on individual response(s) or blood level(s). It may be necessary to use dosages outside these ranges in some cases, as will be apparent to those of ordinary skill in the art. Further, it is noted that the clinician or treating physician will know how and when to interrupt, adjust, or terminate therapy in conjunction with individual patient response.

The term “unit dose” is meant to describe a single dose, although a unit dose may be divided, if desired. About 1 to 10 unit doses of the present invention are typically administered per day, preferably about 2 to 6 doses per day, and more preferably about 4 doses per day.

Although any suitable route of administration may be employed for providing the patient with an effective dosage of the composition according to the methods of the present invention, preferred routes include, for example, oral, rectal, parenteral, intravenous, topical, transdermal, subcutaneous, intramuscular, and like forms of administration may be employed. More preferred routes include oral or topical administration. Suitable dosage forms include tablets, troches, dispersions, suspensions, solutions, capsules, patches, suppositories, and the like, although oral and topical dosage forms are preferred. One skilled in the art would readily know that certain embodiments may or may not be suitable for a certain route of administration due to chemical and/or biological incompatibilities and would use embodiments suitable for the desired route of administration.

The pharmaceutical compositions used in the methods of the present invention include the active ingredients described above, and may also contain pharmaceutically acceptable carriers, excipients and the like, and optionally, other therapeutic ingredients. The active ingredients used in the methods and compositions can be administered individually, as a single composition that contains all the ingredients, or in any combination thereof.

The term “pharmaceutically acceptable salt” refers to a salt prepared from pharmaceutically acceptable non-toxic acids or bases including inorganic or organic acids. Examples of such inorganic acids are hydrochloric, hydrobromic, hydroiodic, sulfuric, and phosphoric. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic. Examples of such inorganic bases, for potential salt formation with the sulfate or phosphate compounds of the invention, include metallic salts made from aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (Nmethylglucamine), and procaine.

The compositions for use in the methods of the present invention may be prepared in various formulations, such as suspensions, solutions and elixirs; aerosols; or carriers such as starches, sugars, microcrystalline cellulose, diluents, granulating agents, lubricants, binders, disintegrating agents, and the like, in the case of oral solid preparations (such as powders, capsules, and tablets), with the oral solid preparations being preferred over the oral liquid preparations. The most preferred oral solid preparations are tablets.

Because of their ease of administration, tablets and capsules represent the most advantageous oral dosage unit form, in which case solid pharmaceutical carriers are employed. If desired, tablets may be coated by standard aqueous or nonaqueous techniques.

Pharmaceutical compositions for use in the methods of the present invention may be presented as discrete units such as capsules, cachets, tablets, or aerosol sprays, each containing a predetermined amount of the active ingredient, as a powder, stick, or granules, as creams (e.g., a conditioner), pastes, gels, lotions, syrups, ointments, sponges or cotton applicators, or as a solution or a suspension in an aqueous liquid, a non-aqueous liquid, an oil-in-water emulsion, or a water-in-oil liquid emulsion.

Such compositions may be prepared by any of the methods of pharmacy, but all methods include the step of bringing into association the carrier with the active ingredient which constitutes one or more necessary ingredients. In general, the compositions are prepared by uniformly and intimately admixing the active ingredient with liquid carriers or finely divided solid carriers or both, and then, if necessary, shaping the product into the desired presentation.

For example, a tablet may be prepared by compression or molding, optionally, with one or more accessory ingredients. Compressed tablets may be prepared by compressing in a suitable machine the active ingredient in a free-flowing form such as powder or granules, optionally mixed with a binder, lubricant, inert diluent, surface active or dispersing agent. Molded tablets may be made by molding, in a suitable machine, a mixture of the powdered compound moistened with an inert liquid diluent. Desirably, each unit dose, i.e., tablet, cachet or capsule, contains from about 1 mg to 2,000 mg of the active ingredient, preferably about 200 mg to 1,600 mg, and more preferably about 600 mg to 1,000 mg of the composition.

The compounds for use in the methods of the present invention may also be administered topically. Topical administration advantageously helps thicken the epidermis. Because of its ease of administration, a cream, lotion, or ointment represents the most advantageous topical dosage form, in which case liquid pharmaceutical carriers may be employed in the composition. These creams, lotions, or ointments, may be prepared as rinse-off or leave-on products. Each of these forms is well understood by those of ordinary skill in the art, such that dosages may be easily prepared to incorporate the pharmaceutical composition of the invention.

In addition to the common dosage forms set out above, the compounds of the invention can also be administered by controlled-release means or delivery devices that are well known to those of ordinary skill in the art, such as those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, the disclosures of which are incorporated herein by reference. Preferred controlled-release means are disclosed by: U.S. Pat. Nos. 5,427,798 and 5,486,362; WO 9404138; CA 1239034; and European Patent Application Nos. 467488 and 171457, all of which are incorporated herein by reference. These dosage forms can be used to provide slow or controlled-release of one or more of the active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, that are adapted for controlled-release are encompassed by the present invention.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

### EXAMPLES

The invention is further defined by reference to the following examples describing in detail the preparation of the compound and the compositions used in the methods of the present invention, as well as their utility. The examples are representative, and they should not be construed to limit the scope of the invention.

### Example 1: Capsules

A large number of unit capsules were prepared by filling standard two-piece hard gelatin capsules each with the desired amount of powdered active ingredient as described above, 150 milligrams of lactose, 50 milligrams of cellulose, and 6 milligrams magnesium stearate.

### Example 2: Soft Gelatin Capsules

A mixture of active ingredient in a digestible oil such as soybean oil, lecithin, cottonseed oil or olive oil is prepared and injected by means of a positive displacement pump into gelatin to form soft gelatin capsules containing the desired amount of the active ingredient. The capsules are washed and dried for packaging.

### Examples 3-11: Pharmaceutical Compositions

Various pharmaceutical formulations containing the ingredients as described below were prepared for the management of the connective tissue ailments of this invention. The formulation variations located in these examples do not provide for all possible combinations. Those skilled in the art would readily know how to combine the various ingredients to maximize the efficacy of the compositions when treating a particular ailment.

### Example 3

| Ingredient | Amount | |
|---|---|---|
| Vitamin A | 2500 | mg |
| Beta Carotene | 1500 | IU |
| Vitamin C | 800 | mg |
| Vitamin E Natural | 280 | IU |
| Zinc Oxide | 24 | mg |
| Copper | 0.6 | mg |
| Green Tea Extract | 192 | mg |
| Echinacea Root Powder | 192 | mg |
| Gentian Root Powder | 192 | mg |
| Golden Seal Root Powder | 192 | mg |
| Myrrh Gum | 192 | mg |
| Poria Cocos | 192 | mg |
| Milk Thistle Seed Extract | 158 | mg |
| N-Acetyl Cysteine | 90 | mg |
| Rosemary Leaf Extract 5:1 | 160 | mg |
| Yellow Duck Root Powder | 150 | mg |
| Glucosamine HCI | 65 | mg |
| L-Gycine | 75 | mg |
| Oreganox ™ | 100 | mg |
| Quercetin | 100 | mg |
| Hawthorn Berry Extract | 80 | mg |
| Wild Yam Root Powder | 80 | mg |
| Bee Pollen | 75 | mg |
| FoTI Root Powder | 58 | mg |
| Royal Jelly Concentrate | 50 | mg |
| Grape Seed Extract | 20 | mg |
| Ginkgo Biloba Leaf Extract | 4 | mg |
| Pomegranate Extract | 5 | mg |
| Biotin | 300 | mcg |
| Selenium | 80 | mcg |
| Essential Fatty Acid Complex** | 150 | mg |

**The Essential Fatty Acid Complex comprise combinations of oleic acid, linoleic acid, gamma linoleic acid, alpha linoleic acid, eicosapentaenoic acid, and docosahexaenoic acid.

### Example 4

| Ingredient | Amount | |
|---|---|---|
| Vitamin A | 1500 | mg |
| Vitamin B2 | 10 | mg |
| Vitamin B6 | 15 | mg |
| Niacin | 15 | mg |
| Zinc | 20 | mg |
| Biotin | 300 | mcg |
| L Arginine HCL | 150 | mg |
| L Alanine | 100 | mg |
| L-Glycine | 75 | mg |
| White Willow Bark | 100 | mg |
| Shark Cartilage | 100 | mg |
| Alpha Lipoic Acid | 80 | mg |
| Cayenne Pepper | 50 | mg |
| Pomegranate Extract | 5 | mg |
| Melatonin | 1 | mg |
| Glucosamine Sulfate | 100 | mg |
| Oreganox ™ | 75 | mg |
| L-Carnitine | 40 | mg |
| Essential Fatty Acids Complex** | 85 | mg |
| Coenzyme Q 10 | 500 | mcg |

**The Essential Fatty Acid Complex comprise combinations of oleic acid, linoleic acid, gamma linoleic acid, alpha linoleic acid, eicosapentaenoic acid, and docosahexaenoic acid.

### Example 5

| Ingredient | Amount | |
|---|---|---|
| Vitamin A | 4000 | IU |
| Beta Carotene | 2500 | IU |
| Vitamin B-1 | 25 | mg |
| Vitamin B-2 | 25 | mg |
| Vitamin B-3 | 50 | mg |
| Vitamin B-5 | 25 | mg |
| Vitamin B-6 | 50 | mg |
| Biotin | 300 | mcg |
| Vitamin C | 300 | mg |
| Folic Acid | 400 | mcg |
| Vitamin E Natural | 400 | IU |
| Calcium | 52 | mg |
| Magnesium | 200 | mg |
| Zinc | 15 | mg |
| Glucosamine HCI | 65 | mg |
| L-Lysine HCI | 250 | mg |
| L-Glycine | 250 | mg |
| L-Proline | 500 | mg |
| Alpha Lipoic Acid | 50 | mg |
| Silica | 28 | mg |
| Grape Seed Extract | 60 | mg |
| Selenium | 200 | mcg |
| Lecithin | 75 | mg |
| Essential Fatty Acid Complex** | 150 | mg |
| Burdock Root Powder | 83 | mg |
| Yellowdock Powder | 97 | mg |
| Chromium | 100 | mcg |
| Pomegranate Extract | 2 | mg |
| Oreganox ™ | 100 | mg |
| Aloe Vera Powder | 75 | mg |
| Coenzyme Q 10 | 800 | mcg |

**The Essential Fatty Acid Complex comprise combinations of oleic acid, linoleic acid, gamma linoleic acid, alpha linoleic acid, eicosapentaenoic acid, and docosahexaenoic acid.

### Example 6

| Ingredient | Amount | |
|---|---|---|
| Vitamin A | 2500 | mg |
| Vitamin B-3 | 60 | mg |
| Vitamin B-6 | 20 | mg |
| Vitamin C | 120 | mg |
| Vitamin E | 60 | IU |
| Chromium Picolinate | 200 | mcg |
| Zinc | 18 | mg |
| Copper | 800 | mg |
| Methyl Sulfonylmethane | 250 | mg |
| Garcinia Cambogia | 250 | mg |
| Essential Fatty Acid Complex** | 150 | mg |
| Inositol | 100 | mg |
| L-Lysine | 125 | mg |
| N-Acetyl Glucosamine | 100 | mg |
| L-Proline | 100 | mg |
| Cayenne | 100 | mg |
| L-Glycine | 75 | mg |
| Choline | 50 | mg |
| N-Acetyl Cysteine | 50 | mg |
| Glucosamine Sulfate HCI | 45 | mg |
| Quercetin | 20 | mg |
| Grape Seed Extract | 15 | mg |
| Selenomethionine | 50 | mg |

**The Essential Fatty Acid Complex comprise combinations of oleic acid, linoleic acid, gamma linoleic acid, alpha linoleic acid, eicosapentaenoic acid, and docosahexaenoic acid.

### Example 7

| Ingredient | Amount | |
|---|---|---|
| Vitamin A Palmitate | 4000 | IU |
| Niacinamide | 60 | mg |
| Vitamin B-6 (Pyridozine HCL) | 20 | mg |
| Vitamin C | 120 | mg |
| Vitamin E Natural | 60 | IU |
| Zinc (amino acid chelate) | 18 | mg |
| N-cetyl D-Glucosamine | 100 | mg |
| L-Lysine HCL | 125 | mg |
| L-Proline | 100 | mg |
| Grape Seed Extract | 15 | mg |
| Glucosamine Sulfate | 1200 | mg |
| N-Acetyl Cysteine | 50 | mg |
| Quercetin | 20 | mg |
| Copper | 0.8 | mg |
| Selenomethionine | 50 | mcg |
| Garcenia | 250 | mg |
| Essential Fatty Acid Complex** | 150 | mg |
| L-Glycine | 75 | mg |
| Chromium Picolinate | 200 | mcg |
| Choline | 50 | mg |
| Cayenne | 100 | mg |
| Inositol | 100 | mg |
| Phosphatidyl Choline | 45 | mg |
| Inositol | 45 | mg |

**The Essential Fatty Acid Complex comprise combinations of oleic acid, linoleic acid, gamma linoleic acid, alpha linoleic acid, eicosapentaenoic acid, and docosahexaenoic acid.

### Example 8

| Ingredient | Amount | |
|---|---|---|
| Vitamin A Palmitate | 4000 | IU |
| Beta Carotene | 2500 | IU |
| Vitamin B1 (Thiamine | 25 | mg |
| Vitamin B-2 (Riboflavin) | 25 | mg |
| Vitamin B-3 (Niacin) | 50 | mg |
| Vitamin B-5 (Pantothenic Acid) | 25 | mg |
| Vitamin B-6 (Pyridozine HCL) | 50 | mg |
| Biotin | 300 | mcg |
| Vitamin C | 300 | mg |
| Folic Acid | 400 | mcg |
| Vitamin E Natural | 400 | IU |
| Calcium (Ascorbate) | 62 | mg |
| Magnesium (Oxide) | 200 | mg |
| Zinc (Ascorbate) | 15 | mg |
| Burdock Root Powder | 83 | mg |
| Glucosamine HCL | 65 | mg |
| L-Lysine HCL | 250 | mg |
| L-Proline | 500 | mg |
| Yellow Dock Root Powder | 97 | mg |
| Silica | 28 | mg |
| Grape Seed Extract | 50 | mg |
| Selenium | 200 | mcg |
| Chromium Picolinate | 100 | mcg |
| L-Glycine | 250 | mg |
| Alpha Lipoic Acid | 50 | mg |
| Lecithin | 75 | mg |
| Pomegranate Extract | 2 | mg |
| Oreganox | 100 | mg |
| Aloe Vera Powder | 75 | mg |
| Coenzyme Q-10 | 800 | mcg |
| Essential Fatty Acid Complex** | 150 | mg |
| Phosphatidyl Choline | 45 | mg |
| Inositol | 45 | mg |

**The Essential Fatty Acid Complex comprise combinations of oleic acid, linoleic acid, gamma linoleic acid, alpha linoleic acid, eicosapentaenoic acid, and docosahexaenoic acid.

### Example 9

| Ingredient | Amount | |
|---|---|---|
| Vitamin A Palmitate | 2500 | IU |
| Beta Carotene | 1500 | IU |
| Vitamin C | 800 | mg |
| Vitamin E | 280 | IU |
| Quercetin | 85 | mg |
| Zinc Oxide | 24 | mg |
| Copper (chelate) | 0.6 | mg |
| Yellow Dock Root Powder | 150 | mg |
| Green Tea Extract | 192 | mg |
| Echinacea Root Powder | 192 | mg |
| Gentain Root Powder | 192 | mg |
| Golden Seal Root Powder | 192 | mg |
| Myrrh Gum | 192 | mg |
| Poria Cocos | 192 | mg |
| Golden Seal Root Powder | 158 | mg |
| N-Acetyl Cysteine | 90 | mg |
| Rosemary Leaf Extract | 160 | mg |
| Hawthorn Berry Extract | 65 | mg |
| Wild Yam Root Powder | 80 | mg |
| Grape Seed Extract | 20 | mg |
| Ginkgo Biloba Leaf Extract | 4 | mg |
| Selenium | 80 | mcg |
| Glucosamine HCL | 65 | mg |
| L-Glycine | 75 | mg |
| Oreganox | 100 | mg |
| Bee Pollen | 75 | mg |
| FoTi Root Powder | 58 | mg |
| Royal Jelly Concentrate | 25 | mg |
| Pomegranate Extract | 5 | mg |
| Biotin | 300 | mcg |
| Essential Fatty Acid Complex** | 125 | mg |
| Choline | 75 | mg |
| Lecithin | 25 | mg |
| Phosphatidyl Choline | 45 | mg |
| Inositol | 45 | mg |

**The Essential Fatty Acid Complex comprise combinations of oleic acid, linoleic acid, gamma linoleic acid, alpha linoleic acid, eicosapentaenoic acid, and docosahexaenoic acid.

### Example 10

| Ingredient | Amount | |
|---|---|---|
| Vitamin A Palmitate | 4000 | IU |
| Niacinamide | 80 | mg |
| Vitamin B-6 (Pyridozine HCL) | 20 | mg |
| Vitamin C | 400 | mg |
| Vitamin E Natural | 100 | IU |
| Zinc (Ascorbate) | 24 | mg |
| N-cetyl D-Glucosamine | 160 | mg |
| L-Lysine HCL | 320 | mg |
| L-Proline | 360 | mg |
| Grape Seed Extract | 30 | mg |
| Selenium | 200 | mcg |
| Glucosamine Sulfate | 1200 | mg |
| N-Acetyl Cysteine | 120 | mg |
| Quercetin | 80 | mg |
| Manganese Ascorbate | 12 | mg |
| Copper | 1.6 | mg |
| Selenomethionine | 80 | mcg |
| Beet Root Powder | 135 | mcg |
| Essential Fatty Acid Complex** | 150 | mg |
| Phosphatidyl Choline | 45 | mg |
| L-Glycine | 45 | mg |
| Inositol | 45 | mg |

**The Essential Fatty Acid Complex comprise combinations of oleic acid, linoleic acid, gamma linoleic acid, alpha linoleic acid, eicosapentaenoic acid, and docosahexaenoic acid.

### Example 11: Tablets

A large number of tablets were prepared by conventional procedures so that the dosage unit included: the desired amount of active ingredient as described herein, 50 milligrams of red beet root powder, 12 milligrams of stearic acid, 10.95 milligrams of sorbitol, 3 milligrams of acdisol, 1 milligram of magnesium stearate, and 1 milligram of syloid. Appropriate coatings may be applied to increase palatability or delay absorption. A specific therapeutic formulation of the pharmaceutical composition prepared in accordance with the invention is set forth in the table below:

| | Weight | | Chemical or |
|---|---|---|---|
| | Percent | Amount | Scientific Name |
| Ingredient | (% w/w) | (mg) | (if different) |
| N-Acetylglucosamine | 17.1 | 140 | N-Acetyl D- |
| | | | Glucosamine |
| Vitamin C (81.2% Ascorbic | 15 | 123.2 | |
| Acid) | | | |
| L-Lysine (80%) | 12.2 | 100 | L-Lysine hydro- |
| | | | chloride |
| L-Proline | 11 | 90 | |
| D-Glucosamine Sulfate | 6.5 | 53.3 | |
| (75%) | | | |
| Chondroitin Sulfate (80%) | 6.1 | 50 | |
| Vitamin E Succinate | 4.3 | 39.7 | D-α tocopheryl acid |
| | | | succinate |
| Zinc monomethionine (20%) | 3.7 | 30 | Zinc DL-methionine |
| N-Acetyl Cysteine | 3.7 | 30 | |
| Manganese Ascorbate (13% | 2.8 | 23.1 | |
| Mn) | | | |
| Vitamin B₃ Niacinamide | 2.4 | 20 | Niacinamide |
| Quercetin Powder | 2.4 | 20 | Quercetin dihydrate |
| Grape Seed Extract | 0.9 | 7.5 | Proanthocyanidin |
| Pyridoxal 5 Phosphate-Co | 0.6 | 5 | P-5-P monohydrate |
| B₆ | | | |
| Selenomethionine (0.5%) | 0.5 | 4 | L-selenomethionine |
| Vitamin A Palmitate | 0.5 | 4 | |
| (500,000 IU/GR) | | | |
| Copper Sebacate (14%) | 0.4 | 2.9 | |
| Red beet root powder | 6.1 | 50 | Beta vulgaris rubra |
| Stearic acid | 1.5 | 12 | |
| Sorbitol | 1.3 | 11 | |
| Acdisol | 0.4 | 3 | Microcrystalline |
| | | | cellulose |
| Coconut oil | 0.1 | 1 | Magnesium stearate |
| Syloid | 0.1 | 1 | Silicon dioxide |
| | | | (amorphous) |

These tablets are an example of one embodiment of a unit dose according to the present invention.

Various modifications of the invention in addition to those shown and described herein will be apparent to those skilled in the art from the foregoing description. Such modifications are also intended to fall within the scope of the appended claims. The foregoing disclosure includes all the information deemed essential to enable those skilled in the art to practice the claimed invention.

### Examples 12-15: Testing of the Product

The tablets of Example 11 were administered to test 73 female subjects to determine the effects on the elasticity, firmness, and presence of fine lines and wrinkles of the skin. A seven day conditioning period was used prior to initiation of the study, where subjects were instructed to discontinue use of all moisturizing products, sun screens and liquid make-ups, and to avoid excessive UV exposure and tanning salons. Subjects were permitted to use their current eye, powder blush, and lip products, and non-moisturizing soap.

Test subjects not in the control group, which consumed placebo tablets, consumed two (2) tablets of the test material of Example 11 daily with meals. Before, and after two (2) and five (5) weeks of tablet use, the subjects were measured as described below. Before measurements were taken, all subjects were allowed to equilibrate for thirty minutes at approximately 68° F. and 44 percent relative humidity. At each measurement phase, three Corneometer readings, a negative impression using Silflo replicating material, and three Ballistometer and Cutometer readings were made on the test sites indicated below.

A total of 65 subjects completed the study, as 7 discontinued the study for unrelated reasons and 1 developed a rash for 5 days. There were 12 subjects in the control group and 53 using the tablets.

### Example 12: Image Analysis

The texture of the skin, fine lines, and wrinkles were assessed by taking Silflo replicas of the periorbital area (crow's feet) at each of the three test times. These negative impressions, or Silflo replicas, were illuminated at a precisely defined angle of 35° to create shadows for analysis by shades of gray. The skin topography is defined by the: (a) number of wrinkles; (b) total area of wrinkles; (c) total length of wrinkles; (d) mean length of wrinkles; and (e) mean depth of wrinkles. The type of wrinkles was determined on the basis of depth, length, and area.

As indicated in Table I below, the number of wrinkles were significantly reduced by 34 percent (p<0.01) and the number of fine lines by 34 percent (p<0.06) as a result of 5 weeks using the test material.

TABLE I
Number of Wrinkles and Fine Lines

| | Number of Wrinkles | | | | Number of Fine Lines | | | |
|---|---|---|---|---|---|---|---|---|
| | Mid-Baseline | | Final-Baseline | | Mid-Baseline | | Final-Baseline | |
| | Control | Treated | Control | Treated | Control | Treated | Control | Treated |
| Average | −3 | −7 | −3 | −15 | −5 | −4 | −6 | −12 |
| Standard Deviation | 9 | 13 | 13 | 12 | 6 | 10 | 14 | 10 |
| p value | p < 0.41 | | p < 0.01 | | p < 0.96 | | p < 0.06 | |
| % Diffe Baseline | −11% | −19% | −6% | −40% | −14% | −24% | −9% | −43% |

| | | | | |
|---|---|---|---|---|
| Total % Difference (T − C) | −8% | −34% | −10% | −34% |

T = Treated
C = Control

Example 12 indicates that use of tablets prepared according to the invention result in a 10 percent decrease in appearance of wrinkles and an 8 percent decrease in fine lines after only 2 weeks of treatment, and a decrease of 34 percent in both wrinkles and fine lines after 5 weeks. Additionally, the observed degree of improvement is a function of the length of treatment as indicated above. This strongly suggests the treatment has imparted an improved skin infrastructure by beneficially affecting the dermis of the skin.

### Example 13: Ballistometer

The Ballistometer is an instrument designed to evaluate in vivo, in a non-invasive manner, the viscoelastic properties of the skin. It analyzes the bounce pattern displayed by a probe that is allowed to impact on the skin. The kinetic energy of the probe striking the skin is stored by the elastic components of the skin and released back to make the probe rebound to a lower height. The height to which the probe will rebound depends upon the amount of stored energy lost in shear viscosity within the skin.

The capacity of the skin to absorb mechanical energy may thus be measured. Although it is unclear exactly which layer, or layers, of the skin are responsible, the mechanical properties of the dermis/epidermis layers are controlled by the density and geometry of the network of collagen fibers. It is believed the Ballistometer describes mostly the tissues underlying the stratum corneum.

Tests were conducted with the Ballistometer on one randomly chosen side of the face, slightly below the cheek bone area. The height of first rebound and the coefficient of restitution (“COR”) were measured. The COR is the ratio of the first to the second rebound. Table II, below, indicates that the COR decreases by 10 percent (p<0.11) and the height of the first rebound reduced by 18 percent (p<0.02) as a result of 5 weeks use of the product. This indicates that less of the energy of the striking probe was restored, thus, a greater amount of energy was dissipated in the skin. This suggests the skin became softer and more yielding during the test period.

TABLE II
Ballistometer Readings

| | Height of First Rebound (mm) | | | | Coefficient of Restitution | | | |
|---|---|---|---|---|---|---|---|---|
| | Mid-Baseline | | Final-Baseline | | Mid-Baseline | | Final-Baseline | |
| | Control | Treated | Control | Treated | Control | Treated | Control | Treated |
| Average | −0.16 | −0.06 | 0.49 | 0.06 | −0.02 | 0.00 | 0.01 | 0.00 |
| Standard Deviation | 0.41 | 0.48 | 0.52 | 0.51 | 0.03 | 0.02 | 0.03 | 0.03 |
| p value | p < 0.56 | | p < 0.02 | | p < 0.06 | | p < 0.11 | |
| % Difference from Baseline | −6% | 0% | 22% | 4% | −12% | −0% | 12% | 2% |

| | | | | |
|---|---|---|---|---|
| Total % Difference (T − C) | 6% | −18% | 12% | −10% |

T = Treated
C = Control

### Example 14: Cutometer

The Cutometer is a commercially available instrument (Courage & Khazaka, Germany) designed to measure the mechanical properties of the skin in a non-invasive manner. It measures the vertical deformation of the skin's surface when pulled by vacuum suction (500 mm Hg) through the small aperture (2 mm) of a probe and the depth of penetration of the skin into the probe optically with an accuracy of 0.01 mm. The probe is attached to a computer, which completely controls probe operation and plots skin deformation as a function of time. From this curve, a number of variables can be extrapolated to estimate the elastic, viscoelastic, and purely viscous behavior of the skin.

The following parameters were recorded: (a) the immediate distension (Uₑ), measured at 0.1 seconds; (b) the delayed distension (Uᵥ); (c) the final distension (U_{f}), measured at 10 seconds; and (d) immediate retraction (Uᵣ). The deformation parameters are extrinsic parameters dependent on skin thickness, and a variety of biologically important ratios were calculated: (a) Uᵣ/U_{f}, a measure of net elasticity of the skin; (b) Uᵣ/Uₑ, the biological elasticity, or measurement of the ability of the skin to regain its initial configuration after deformation; and (c) Uᵥ/Uₑ, the viscoelastic to elastic ratio, where an increase in this ratio indicates and increase in the viscoelastic portion of the deformation and/or a relative decrease of the elastic portion.

Tests were conducted using a Cutometer on both sides of the face on the cheek area. Table III, below, indicates that the delayed distension (Uᵥ) decreased a significant 16 percent (p<0.04) after 5 weeks of treatment. This parameter reflects viscoelastic properties of the skin and, thus, the behavior of the dermis. After 5 weeks, there were no statistically significant changes in Uₑ, the immediate distension, which is primarily affected by the moisture content and mechanical properties of the stratum corneum.

TABLE III
Cutometer Readings

| | U_{f}(mm) | | | | Uₑ(mm) | | | |
|---|---|---|---|---|---|---|---|---|
| | Mid-Baseline | | Final-Baseline | | Mid-Baseline | | Final-Baseline | |
| | Control | Treated | Control | Treated | Control | Treated | Control | Treated |
| Average | 0.071 | 0.040 | 0.026 | 0.020 | 0.046 | 0.021 | 0.008 | 0.009 |
| Standard Deviation | 0.038 | 0.058 | 0.058 | 0.049 | 0.028 | 0.042 | 0.048 | 0.043 |
| p value | p < 0.11 | | p < 0.71 | | p < 0.08 | | p < 0.96 | |
| % Difference from Baseline | 39% | 20% | 16% | 11% | 36% | 16% | 11% | 10% |
| Total % Difference (T − C) | −19% | | −5% | | −20% | | −1% | |
| | Uᵥ(mm) | | | | Uᵣ(mm) | | | |
| | Mid-Baseline | | Final-Baseline | | Mid-Baseline | | Final-Baseline | |
| | Control | Treated | Control | Treated | Control | Treated | Control | Treated |
| Average | 0.026 | 0.020 | 0.018 | 0.010 | 0.033 | 0.017 | 0.013 | 0.008 |
| Standard Deviation | 0.015 | 0.018 | 0.015 | 0.011 | 0.018 | 0.027 | 0.030 | 0.023 |
| p value | p < 0.27 | | p < 0.04 | | p < 0.09 | | p < 0.55 | |
| % Difference from Baseline | 51% | 39% | 34% | 19% | 48% | 26% | 19% | 15% |
| Total % Difference (T − C) | −12% | | −16% | | −22% | | −5% | |
| | Uᵣ/Uₑ | | | | Uᵥ/Uₑ | | | |
| | Mid-Baseline | | Final-Baseline | | Mid-Baseline | | Final-Baseline | |
| | Control | Treated | Control | Treated | Control | Treated | Control | Treated |
| Average | 0.004 | 0.034 | 0.042 | 0.027 | 0.017 | 0.063 | 0.092 | 0.048 |
| Standard Deviation | 0.105 | 0.064 | 0.062 | 0.064 | 0.073 | 0.078 | 0.132 | 0.073 |
| p value | p < 0.21 | | p < 0.45 | | p < 0.08 | | p < 0.13 | |
| % Difference from Baseline | 2% | 7% | 9% | 6% | 8% | 19% | 28% | 16% |

| | | | | |
|---|---|---|---|---|
| Total % Difference (T − C) | 5% | −3% | 12% | −12% |

| Uᵣ/Uᵣ | |
|---|---|
| Mid-Baseline | Final-Baseline |

| | Control | Treated | Control | Treated |
|---|---|---|---|---|
| Average | 0.024 | 0.014 | 0.012 | 0.003 |
| Standard Deviation | 0.034 | 0.040 | 0.036 | 0.037 |
| p value | p < 0.47 | | p < 0.46 | |
| % Difference from Baseline | 6% | 4% | 3% | 1% |

| | | |
|---|---|---|
| Total % Difference (T − C) | −2% | −2% |

T = Treated
C = Control

### Example 15: Corneometer

The general appearance of soft, smooth skin depends largely on the presence of an adequate amount of water in the stratum corneum. The Corneometer is a commercially available instrument (Courage & Khazaka, Germany) to measure the changes in capacitance of the skin resulting from changes in the degree of hydration. It is particularly sensitive to low levels of hydration, and uses measurements of arbitrary units of skin hydration (H) to express capacitance.

Tests were conducted using a Corneometer on both sides of the face on the cheek area. Changes in moisture content of the stratum corneum occur rapidly due to changes in the environment, including hydration from the use of moisturizing agents and humectants. Thus, the measurements with the Ballistometer and Cutometer indicate changes occurred in deeper layers of the skin, rather than the superficial stratum corneum. Table IV shows no significant changes in the hydration of the stratum corneum following 2 weeks (p<0.84) and 5 weeks (p<0.67) of product use.

TABLE IV
Corneometer Readings

| | Skin Hydration (H) | | | |
|---|---|---|---|---|
| | Mid-Baseline | | Final-Baseline | |
| | Control | Treated | Control | Treated |
| Average | −5 | −7 | −8 | −4 |
| Standard Deviation | 6 | 7 | 5 | 7 |
| p value | p < 0.84 | | p < 0.67 | |
| % Difference from | −7% | −10% | −12% | −6% |
| Baseline | | | | |

| | | |
|---|---|---|
| Total % Difference (T − C) | −3% | 6% |

T = Treated
C = Control

### Examples 16-18: Testing of Various Embodiments of the Invention

The effectiveness of an orally ingested product according to the invention was tested for its ability to reduce the appearance of cellulite in the thigh area. A total of eighteen (18) female subjects ranging in age from 32 to 65 years of age were selected to evaluate the composition of the invention. All subjects exhibited visible signs of moderate or greater cellulite (Grade 2 or higher); had the absence of any visible skin disease(s) which might be confused with a skin reaction from the test material; were in general good health with no known allergies, especially to cosmetic or toiletry products; had no evidence of acute or chronic disease; had completed a medical history form, as well as understood and signed an Informed Consent form; were dependable and capable of following directions; were not pregnant or lactating; were not on any diet or weight reduction program; and were not on any regular exercise program (immediately prior to or during the course of the study).

At baseline, each subject received a visual examination conducted by a qualified technician, and had a skin replica made of the area considered most representative of the overall appearance of cellulite. Subjects were tested on the right or left thigh. The test sites were carefully demarcated to ensure subsequent evaluations were made on the same test areas. The test sites were examined by a trained observer and scored for the degree of cellulite at each clinic visit according to the following scale:

0=No visible cellulite

1=Very little visible cellulite, no dimpling

2=Visible cellulite, evidence of shallow dimpling

3=Easily visible cellulite, moderate to pronounced dimpling

4=Extremely visible cellulite, heavy and deep dimpling

Note: Half-point increments could be used to better define observed levels of cellulite.

Replicas were completed with the subject in a standing position. Silicone replicas (2 cm×5 cm) were obtained at the baseline and week 6 using the following materials: Silflo® Silicone Impression material, (commercially available from Flexico, of England), Silflo® Universal Accelerator, weighing boat, and spatula.

The panel was divided into two groups, nine (9) subjects per group. The first group, Group A, took two (2) Youth Builder™ supplements, twice daily, preferably with meals (morning and evenings). The second group, Group B, took two (2) Youth Builder™ supplements, preferably with meals (morning and evenings) and one (1) Garcinia tablet twice daily, preferably with meals (morning and evenings).

The Youth Builder™ supplements (commercially available from Murad Inc., of El Segundo, Calif.) contained the following ingredients:

| Ingredient | Percent (w/w) |
|---|---|
| Vitamin A (palmitate) | 0.33 |
| Niacinamide | 1.67 |
| Vitamin B-6 (from pyrodoxine HCL) | 0.42 |
| Vitamin C (from magnesium ascorbate) | 8.33 |
| Vitamin E (d-alpha tocopheryl succinate) | 1.75 |
| N-acertyl D-glucosamine | 3.33 |
| L-proline | 7.5 |
| L-lysine (HCL) | 6.67 |
| Glucosamine sulfate | 11.7 |
| N-acetyl cysteine | 3.33 |
| Quercetin | 2.50 |
| Grape seed extract (38.4%) | 1.67 |
| Zinc (Opti-Zinc) | 0.63 |
| Manganese (ascorbate) | 2.50 |
| Copper (sebacate) | 0.70 |
| Selenomethionine (L-selenomethionine) | 0.08 |
| Beet root powder | 0.01 |
| Dicalcium phosphate | 15.6 |
| Microcrystalline cellulose | 1.67 |
| Stearic acid | 23.3 |
| Magnesium stearate | 1 |
| Silica | 1 |
| Croscarmellose sodium | 4.2 |
| Talc | 0.21 |
| Pharmaceutical glaze | |

The Garcinia tablets, 365 Murad™, (commercially available from Murad, Inc., of El Segundo Calif.) contained citrin® (garcinia cambogia, 2000 mg (yielding 100 mg of (−) hydroxycitric acid as calcium hydroxycitrate), 200 mg L-phenylalanine, and 200 mg chromium.

Subjects were provided with a three (3) week supply of their randomly assigned test product(s) for use at home and were instructed to discontinue the use of their normal anti-cellulite products, to avoid introducing any new products for treating cellulite during the study, and to not be on any diet or weight reduction program or on any regular exercise program immediately prior to or during the course of the study. Each subject was also instructed to keep a diary to document compliance. After three weeks of product usage, the subjects returned to the laboratory for a visual evaluation of the test sites. Another three (3) week supply of their assigned test product(s) and a new diary was then dispensed to each subject. After the second three (3) weeks of product usage (week 6), the subjects returned to the laboratory for a final visual evaluation and to have another skin replica of the test site taken. All diaries were reviewed for compliance.

Seventeen (17) subjects completed the evaluation. The remaining subject discontinued participation due to personal reasons unrelated to product usage. During the study five (5) patients reported minor adverse events such as itching and/or redness, stomach cramps, diarrhea, and constipation. None of the adverse events were serious enough to cause a subject to discontinue the study.

Statistical Analysis of the data was performed using repeated measures ANOVA to determine if any significant (P<0.05) differences were observed between the baseline, the 3 week, and the 6 week post-treatment scoring intervals. Repeated measures ANOVA and dependent t-test were performed using ThinkPoint™ Statistics Menu V.5, for Microsoft Excel V.5 or later (commercially available from ThinkPoint™ Statistics Menu, LLC, Sandy, Utah). If significance was observed, the t-Test (Dependent) was used to compare individual columns of data.

### Example 16: Visual Assessment

Visually-assigned individual cellulite scores were assessed. For Group A (Youth Builder™ supplements alone), one in eight subjects (⅛, 12.5%) exhibited improvement in visually scored cellulite at the 3 week post treatment scoring interval and three of eight subjects (⅜, 37.5%) exhibited improvement at the 6 week post treatment scoring interval when compared to baseline.

For Group B (Youth Builder™ supplements and Garcinia), three of nine subjects (3/9, 33.3%) exhibited improvement in visually scored cellulite at the 3 week post treatment scoring interval and six of nine subjects (6/9, 67.7%) exhibited improvement at the 6 week post treatment scoring interval when compared to baseline.

Although there was a small number of test subjects, certain trends are evident from the statistical analysis. For subjects taking Youth Builder™ supplements alone (Group A) the overall improvements in visually scored cellulite were not significant at P<0.05, but exhibited marginal significance at P<0.089 (Repeated Measures Analysis of Variance). For subjects taking Youth Builder™ supplements and garcinia, repeated measures ANOVA indicated that highly significant (P≦0.008) improvements in visually scored cellulite were obtained between baseline and/or the 3 week and 6 week post treatment scoring intervals. t-Test (Dependent) analyses showed marginal significance (P≦0.081) between baseline and week 3 but highly significant (P≦0.008) improvements between baseline and week 6.

### Example 17: Skin Unevenness

The replicas of the 17 subjects who completed the study were analyzed. A skin surface contour analysis was performed on the replicas. The skin contour analysis was performed with an IBM compatible Pentium III 500 Mhz personal computer with a math co-processor and 128 mb memory running under Windows 98; and a SONY solid state B&W video camera with a 50 mm lens/5 mm extension, ITI OFG frame grabber, and SONY Trinitron monitor. The computer was equipped with OPTIMAS v6.2, Microsoft EXCEL 2000, and StatSoft STATISTICA 5.5 software. Illumination was provided by a structured laser light source consisting of parallel lines projected at 45 degrees from the plane of the replica surface.

The skin contour replica analysis was performed by taking a replica of the thigh surface with Siflo® silastic resin using a specialized frame to orient and support the replica on the skin. The resulting replica is approximately 2 cm by 5 cm. Skin surface elevation or contour, as sampled by the replica, was then determined and the degree of variation of the elevation over the sampled surface was analyzed. The replica surface elevation is measured by the displacement of a projected array of laser light lines spaced at intervals over the replica surface. When viewed from above, the image of the lines on the replica appear displaced from the reference positions in proportion to the contour height of the replica surface.

For the 2 cm by 5 cm replica, five (5) equally spaced laser lines were projected and analyzed. The image analyzer was programmed to determine the location of each displaced projected line at 10 equally spaced locations along the length of the laser line. The displacement (difference between the positions measured for a planar reference and positions measured for the replica sample) was calculated for each of 50 sampling points. The data for each replica constituted a map of the surface elevation at 50 points on the surface. The data was recorded in a raw data file as the spacing parameter. The standard deviation of the elevations was computed as a measure of the unevenness of the surface. To overcome contributions from a general tilt of the replica surface, which does not contribute to the unevenness of the surface, the spacing data was fit to a hypothetical flat surface and the differences between the hypothetical reference surface and the observed map elevations was computed. The standard deviation of these differences called the Standard Deviation of the Residuals (RSTD) is a measure of the unevenness of the contour surface independent of any overall tilt in the replica surface. The unit of measure for the RSTD is millimeters. The RSTD value was recorded in the results file and constituted the main reported datum for the replica.

The following parameters are reported for the skin contour analysis:

SPSTD: Standard deviation of spacing shift, which measures the variation of the surface including overall shape contribution. The smaller the SPSTD the smoother the surface of the skin.

RSTD: Standard deviation of the residuals. The smaller the RSTD the smoother the surface of the skin.

NLARGE: The number of points falling more than a standard deviation above or below the fitted surface. The smaller the NLARGE the smoother the surface of the skin.

Replicas taken from subjects at baseline (T₀) and after the final visit (T₁) were compared.

Changes from baseline were calculated by subtracting the baseline values for each subjects from the final values. The mean changes were tested for significance using the one sample t-test against a value of zero.
t=^{\sqrt{n}·(mean-u}^{₀}⁾/_{std.dev.}where u₀=0.
The t statistic was compared with values of the t distribution for the known degrees of freedom, n-1.

The data showed statistically significant changes (p<0.05) from baseline for all 3 parameters for group A but not group B. The combined groups also exhibited statistically significant changes for the SPSTD and RSTD parameters bust not the NLARGE parameter. The results for groups A and B were also compared using the t-Test for independent groups. While the trend was in the direction of A being more improved than B, the differences were not statistically significant.

Overall, significant improvements in skin unevenness parameters were found for Group A and for the two groups combined.

### Example 18: Skin Texture, Roughness and Wrinkles

The same replicas were also examined for changes in fine texture in the center portion of the 2×5 cm sample. In these replicas, the fine lines run in the long dimension of the sample so the texture assay was made with lighting directed normal to the long dimension. Skin contour analysis for wrinkle texture parameters were performed with an IBM compatible Pentium III 500 Mhz personal computer with a math co-processor and 128 mb memory running Windows 98 and a Cohu Solid State B&W video camera with a 50 mm lens/30 mm extension, ITI OFG frame grabber and SONY Trinitron monitor. The computer was equipped with OPTIMAS v6.2, Microsoft EXCEL 2000, and StatSoft STATISTICA 5.5 software. Illumination was obtained using a collimated light source directed at a 25 angle from the plane of the replica.

The general background gradient of light intensity was adjusted by applying a 2^{nd} order correction in the direction of the light propagation. The shadow texture produced by the oblique lighting of the negative replica was analyzed by two types of assay methods (A and B).

Method A: The luminance was measured along a set of 10 equal length parallel lines (passes) running across the replica parallel to the lighting direction. The variations in luminance (lightness/darkness) were treated as indicative of the roughness and analyzed by the following traditional surface roughness statistics:

Rz: The average maximum difference in luminance value for five equal length segments in each of the 10 lines traversing the sample. The Rz value measures the maximal optical roughness.

Ra: The average deviation of the luminance curve about the mean luminance for the same 10 lines. The Ra value measures the average optical roughness.

The Rz and Ra parameter are reported in the units of brightness (gray levels) ranging from 0 to 255.

Fspace: Measures the distance between markers placed on the lines at luminance changes indicative of fine line spacing (mm).

Fnum: Is the number of fine line markers (per mm).

Method B: The replica image area is divided into 10 equal width bands or sub-areas. Shadow like features are detected in each of these bands according to their luminance values being less than the detection threshold (Threshold Algorithm: Cutoff=that gray level halfway between black and the most probable gray value in the image. The most probable gray value typically characterizes the flat, featureless regions of the replica. Shadows-100.0*sum (ArROIHistogram[Lo..(CUTOFF+1)])/totalpixels). Four parameters were determined from the detected features.

Spacing: The mean distance in millimeters between adjacent detected features (i.e., the spacing between the midpoints of adjacent shadowy features).

Breadth: The average breadth of the detected features in millimeters. This parameter is proportional to the depth of the wrinkle producing the shadow.

Shadows: The percent of the sampled replica area with luminance values less than the detection threshold. This is the relative area of shadows cast by the wrinkles and fine lines in the replica.

Num Wrinkles: The total number of features detected in the 10 bands or sub-areas used to calculate spacing and breadth.

Statistically significant changes were found for Fspace, Fnum, Spacing, and Num Wrinkle parameters. The significant changes more often involved group A than group B. The combined groups also exhibited statistically significant changes in these 4 parameters. Overall, the results suggest a tighter, more compact texture as a result of treatment, especially for group A.

The results of Examples 12-14 show that significant improvements in the skin unevenness parameters were found for Group A (Youth Builder™ supplements alone) and for both groups (A and B) combined. The results of the texture analysis suggest a tighter, more compact texture as a result of treatment, especially for Youth Builder™ supplements alone. The results of the study show significant or near significant improvements in visibly scored cellulite are obtained for both Youth Builder™ supplements alone (more pronounced using skin image analysis) and for Youth Builder™ supplements plus Garcinia (more pronounced using visual scoring of cellulite levels).

Various modifications of the invention in addition to those shown and described herein will be apparent to those skilled in the art from the foregoing description. Such modifications are also intended to fall within the scope of the appended claims. The foregoing disclosure includes all the information deemed essential to enable those skilled in the art to practice the claimed invention.

1-24. (canceled)

25. A pharmaceutical composition for managing a connective tissue disorder in a patient comprising an effective amount of each of the following components:
(i) a sugar compound that is converted to a glycosoaminoglycan in the patient;
(ii) a primary antioxidant component;
(iii) at least one amino acid component;
(iv) at least one transition metal component;
(v) at least one moisturizing agent; and
(vi) at least one fatty acid.

26. The pharmaceutical composition of claim 25, wherein the pharmaceutical composition is adapted for topical administration.

27. The pharmaceutical composition of claim 26, further comprising one or more mono- or poly-hydroxy acids or tannic acid, a mixture thereof, or a pharmaceutically acceptable salt or ester thereof in an amount sufficient to exfoliate at least a portion of the skin.

28. The pharmaceutical composition of claim 26, further comprising hydrogen peroxide in an amount sufficient to cleanse the skin.

29. The pharmaceutical composition of claim 25, wherein the sugar compound that is converted to a glycosoaminoglycan is present in an amount ranging from about 5 to 50 weight percent; the primary antioxidant component is present in an amount ranging from about 5 to 50 weight percent; the amino acid component is present in an amount ranging from about 8 to 60 weight percent; the transition metal component is present in an amount ranging from about 2 to 12 weight percent; the moisturizing agent is present in an amount ranging from about 0.01 to 20 weight percent; and the fatty acid is present in an amount ranging from about 0.01 to 20 weight percent.

30. The pharmaceutical composition of claim 25, wherein the amino acid component is selected from the group consisting of lysine, proline, cysteine, glycine, methionine, and mixtures thereof.

31. The pharmaceutical composition of claim 25, wherein the transition metal component is selected from the group consisting of zinc, manganese, copper, and mixtures thereof.

32. The pharmaceutical composition of claim 25, further comprising (vii) a vascular dilator.

33. The pharmaceutical composition of claim 32, wherein the vascular dilator is selected from the group consisting of extract of ginko biloba, ginsing extract, phenylalanine, and niacin.

34. The pharmaceutical composition of claim 33, wherein the sugar compound that is converted to a glycosoaminoglycan is present in an amount ranging from about 5 to 50 weight percent; the primary antioxidant component is present in an amount ranging from about 5 to 50 weight percent; the amino acid component is present in an amount ranging from about 8 to 60 weight percent; the transition metal component is present in an amount ranging from about 2 to 12 weight percent; the moisturizing agent is present in an amount ranging from about 0.01 to 20 weight percent; the fatty acid is present in an amount ranging from about 0.01 to 20 weight percent; the extract of ginko biloba, if present, is present in an amount ranging from about 5 mg to 300 mg; the ginsing extract, if present, is present in an amount ranging from about 100 mg to 200 mg; the phenylalanine, if present, is present in an amount ranging from about 75 mg to 1500 mg; and the niacin, is present in an amount ranging from about 5 mg to 1500 mg.

35. The pharmaceutical composition of claim 32, wherein the amino acid component is selected from the group consisting of lysine, proline, cysteine, glycine, methionine, and mixtures thereof.

36. The pharmaceutical composition of claim 32, wherein the transition metal component is selected from the group consisting of zinc, manganese, copper, and mixtures thereof.

37. The pharmaceutical composition of claim 25, wherein the at least on moisturizing agent comprises a hydrophilic moisturizing agent and a hydrophobic moisturizing agent.

38. The pharmaceutical composition of claim 37, wherein the sugar compound that is converted to a glycosoaminoglycan is present in an amount ranging from about 5 to 50 weight percent; the primary antioxidant component is present in an amount ranging from about 5 to 50 weight percent; the amino acid component is present in an amount ranging from about 8 to 60 weight percent; the transition metal component is present in an amount ranging from about 2 to 12 weight percent; the hydrophilic moisturizing agent is present in an amount ranging from about 0.01 to 20 weight percent; the hydrophobic moisturizing agent is present in an amount ranging from about 0.01 to 20 weight percent; and the fatty acid is present in an amount ranging from about 0.01 to 20 weight percent.

39. The pharmaceutical composition of claim 37, wherein the amino acid component is selected from the group consisting of lysine, proline, cysteine, glycine, methionine, and mixtures thereof.

40. The pharmaceutical composition of claim 37, wherein the transition metal component is selected from the group consisting of zinc, manganese, copper, and mixtures thereof.
